(19) United States
(12) Patent Application Publication
(10) Pub. No.: US 2004/0071653 A1
(43) Pub. Date: Apr. 15, 2004

(54) Antimicrobial ternary surfactant blend comprising cationic, anionic, and bridging surfactants, and methods of preparing same

(75) Inventors: Daniela T. Bratescu, Glenview, IL (US); Randal J. Bernhardt, Lindenhurst, IL (US)

Correspondence Address: MCANDREWS HELD & MALLOY, LTD, 500 WEST MADISON STREET, SUITE 3400, CHICAGO, IL, 60661

(73) Assignee: Stepan Company, Northfield, IL

(21) Appl. No.: 10/677,422

(22) Filed: Oct. 2, 2003

Related U.S. Application Data
(63) Continuation of application No. 09/661,839, filed on Sep. 14, 2000, now abandoned.

(51) Int. Cl. A61K 31/14; A61K 7/075; A61K 7/08

(52) U.S. Cl. 424/70.24; 424/70.28; 514/642

(57) ABSTRACT

Disclosed are antimicrobial compositions comprising

(a) a quaternary ammonium antimicrobial compound;

(b) an anionic surfactant; and

(c) a bridging surfactant.

Further, further disclosed are a method of preparing the antimicrobial compositions and a method of inhibiting growth of microbial organisms by contacting with the antimicrobial compositions.

### FIELD OF THE INVENTION

The present invention relates to antimicrobial compositions and more specifically to such compositions containing at least one quaternary ammonium antimicrobial compound, at least one anionic surfactant and optionally at least one “bridging surfactant.”. Further, the instant invention relates to a method of preparing the antimicrobial compositions and methods of inhibiting growth of microbial organisms by contacting with the antimicrobial compositions.

### BACKGROUND OF THE INVENTION

Antimicrobial surfactant compositions have been employed in a wide variety of finished consumer end use applications. Among these are detergents, liquid dish cleansers, powdered cleansers and detergents, hard surface cleaners, and personal care formulations, such as shampoos, body washes, and hand soaps.

U.S. Pat. No. 4,264,457 (to DeSoto) discloses various anionic/cationic fabric softening compositions. U.S. Pat. No. 4,576,729 (to Sterling Drug) discloses stable disinfectant laundry detergents. U.S. Pat. No. 4,272,395 (to Lever Brothers) discloses high foaming germicidal detergent compositions. U.S. Pat. No. 5,922,693 (to Colgate-Palmolive) discloses various cleaning compositions containing biostat agents. U.S. Pat. No. 5,441,541 (to Colgate-Palmolive) discloses a wide variety of anionic/cationic complexes. U.S. Pat. No. 45,622,925 (to Procter & Gamble) discloses stable aqueous laundry detergents with improved softening properties.

For other mixed surfactant systems related to antimicrobial compositions, see generally, WO 97/15647; WO 97/28238; WO 97/12022; EP 0852947A2; EP 0916718A1; JP 8333592; and JP 7034079.

Generally, anionic-cationic surfactant mixtures are well known to the art. See generally, U.S. Pat. Nos. 5,441,541, 5,472,455, 5,204,010, 4,790,856, 4,298,480, 3,730,912 (all to The Colgate-Palmolive Company), 5,622,925, 5,607,980, 5,565,145, 4,913,828, 4,659,802, 4,436,653, 4,338,204, 4,333,862, 4,132,680 (all to The Procter & Gamble Co.); also see WO 97/03164, WO 97/12022 and WO 96/37591 (all to The Procter & Gamble Co.), and WO 97/28238 and WO 97/15647 (both to Reckit & Colman, Inc.). Se also, U.S. Pat. Nos. 5,610,187 and 4,247,538 (both to Witco Corp.), 5,344,949 (to Th. Goldschmidt AG), 5,332,854 and 5,324,862 (both to Dai-Ichi Kogoyo Seiyaku Co., Ltd.), 4,273,760 (to National Starch and Chemical), and 4,264,457 (to DeSoto, Inc.). Mixed surfactant systems have also been disclosed in “Mixed Surfactant System”, ACS Symposium Series 501, P. M. Holland and D. N. Rubingh (Jun. 17-19, 1991).

Additionally, there have been many studies and symposia on mixed surfactant systems. See, for example, Scamehorn, J. F., ed., “Phenomena in Mixed Surfactant Systems”, ACS Symposium Series 311, Washington, D.C. (1986). The effects of alkyl groups and oxyethylene groups in nonionic surfactants on the surface tension of anionic-nonionic systems have been described. See Abe et al., J. Colloid Interface Sci., 107, p. 503 (1985); Ogino et al., J. Colloid Interface Sci., 107, p. 509 (1985); and Rosen et al., J. Colloid Interface Sci., 95, 443 (1983). Interaction between betaines and cationic surfactants has also been studied. See Zhu et al., J. Colloid Interface Sci., 108, 423 (1985).

Mixed surfactant systems have shown synergistic improvements in surfactant properties compared to the properties of their individual surfactant components. Synergism increases with the degree of charge difference. Thus, the greatest synergistic surfactant property improvements are realized when mixing anionic and cationic surfactants. See Rosen et al. in “Phenomena in Mixed Surfactant Systems” (Scamehorn, J. F., ed.), ACS Symposium Series 311, Washington, D.C. (1986), pp. 144-162; Zhao et al. in “Phenomena in Mixed Surfactant Systems” (Scamehorn, J. F., ed.) ACS Symposium Series 311, Washington, D.C. (1986) pp. 184-198.

In detergent applications, although in principle any surfactant is suitable, in practice only anionic and nonionic surfactants typically are used. Cationic surfactants, especially quaternary ammonium salts, can decrease detergency and enhance soil redeposition when used in heavy-duty liquid detergents. Consequently, there is a general notion that anionic and cationic surfactants cannot be used in the same formula without loss of efficacy. Similar worries regarding potential loss of efficacy exist when contemplating use of cationic surfactants in hair and skin conditioning applications. Thus, anionic-cationic surfactant mixtures have been used only sparingly in the production of consumer cleaning products and personal care products.

Studies on anionic-cationic systems are recent and few compared to studies on other mixed surfactant systems. However, strong synergism has been exhibited by these systems. Surface activity properties, particularly the critical micelle concentration (cmc), surface tension, and microemulsion behavior (Bourrel et al., Tenside Detergents, 21, 311 (1984)), were the most studied properties. For example, the surface activities of mixed aqueous solutions of sodium dihexylsulfosuccinate with dioctyl(hydroxyethyl)methylammonium chloride and sodium dihexylsulfosuccinate with octyl(hydroxyethyl)dimethylammonium chloride were much higher than those of the single surfactants. See Zao, G., Huoxue Xuebo, 43, 705 (1985) (Ch. Chem. Abstracts 103:184033n). The strong synergistic effect on surface pressure for mixed solutions of cationic and anionic surfactants has been studied quantitatively. When dilute solutions of sodium dodecylsulfate and dodecyltrimethylammonium bromide were mixed, tile surface pressure increased by more than 40 mN/m. Also, the cmc and the minimum surface tension were lower for the mixture than for either the anionic or cationic surfactants alone (Lucassen-Reynders et al., J. Colloid Interface Sci., 81, p. 150 (1981)).

However, mixed anionic-cationic mixtures also have shown antagonistic effects relative to the properties of the individual surfactant components. See Chobanu et al., Izv. Akad. Nauk. Mold. SSR, Ser. Biol. Khim. Nauk., 5, p. 66 (1982). Unlike other mixed surfactant systems, most anionic-cationic surfactant mixtures studied are insoluble or only slightly soluble in water. Hence, practical use of anionic-cationic surfactant mixtures has been very limited in areas where a relatively high concentration of surfactants is needed (see U.S. Pat. No. 5,472,455, to Mehreteab, issued Dec. 5, 1995). Thus, there is a need for soluble anionic-cationic surfactant mixtures.

At present, very few anionic-cationic surfactant mixtures have been found which produce clear solution phases over a wide concentration range at equimolar composition. See generally, Khan, A.; Marques, E.; Spec. Surfactants 1997, 37-80, edited by Robb, I. D. Blackie. Typically, anionic-cationic surfactant mixtures are present as microemulsions, rather than as clear, homogeneous solutions. Usually, the anionic and/or cationic surfactant must be alkoxylated to even maintain such a microemulsion.

Because the probability of synergism between surfactants increases with the strength of interaction, the greatest probability of synergism with anionic surfactants exists in anionic-cationic or anionic-zwiterionic mixtures. See generally, Surfactant and Interfacial Phenomena; Rosen, M.; John Wiley & Sons, Inc. 1989 p. 402. Surfactant performance is gauged by the so-called β value, which is a negative number indicating how much less a system's actual surface tension is compared to its calculated surface tension. Surfactant mixtures exhibiting larger deviations between calculated and actual surface tension perform better; thus, surfactant performance increases with progressively more negative β values. However, with respect to anionic-cationic mixtures, the variations in surfactant type and size that produce progressively more negative β values unfortunately are accompanied by decreasing solubility. Hence anionic-cationic synergism is limited by the formation of an insoluble salt, which typically occurs when the combined number of carbon atoms in the chains of both surfactants totals more than about twenty. See generally, Lomax, E; Specialty Chemicals 1993, v 13 n 4 p 223-227). A method for enhancing the solubility of anionic-cationic surfactant mixtures is therefore needed to allow achieving maximum negative βvalues.

Without being bound by any particular theory, the benefits associated with solubilized anionic/cationic systems are best explained by the theory that surfactant molecules of opposite charge pack more closely to each other in micelles due to the absence of any electrostatic repulsion. This close packing in turn leads to more efficient soil removal. See generally, Lomax, E., supra. Prior art attempts to solubilize anionic-cationic surfactant systems include the use of organic solvents, such as butanol or ethanol. Also, reported is the use of nonionic surfactants as solubilizing agents or incorporation of alkoxy groups into the anionic and/or cationic surfactants. Unfortunately, addition of organic solvents presents a fire hazard. Additionally, addition of nonionic components tends to keep the anionic and cationic surfactant molecules further apart, decreasing the overall efficacy of the system. Once again without being bound by any particular theory, the oppositely charged surfactant molecules are kept further apart due to stearic hindrance and because of the osmotic effects which force water molecules between the two surfactant molecules, diminishing the beneficial effect of closer packing.

In addition, the antimicrobial activity associated with antimicrobial quaternary ammonium compounds is generally lost when such compounds are formulated into disinfectant or sanitizing compositions having an anionic surfactant. Thus, there is a need for compositions having anionic surfactants in combination with quaternary ammonium antimicrobial compounds that display efficacious antimicrobial activity. This need has not previously been met.

### SUMMARY OF THE INVENTION

Alcohols are known to possess low-level broad spectrum germicidal activity. Ethanol, benzyl alcohol, and isopropanol are currently used in disinfecting compounds effective against bacteria Isopropanol, at a concentration of greater than 50% by weight, is the preferred alcohol. Alcohols work by denaturing and precipitating proteins of the microorganism. Alcohols have very low vapor pressures and consequently are quite flammable. Ethyl alcohol is effective against many bacteria only in concentrations exceeding 50% and thus is a hazard in any bactericidal composition since its flash point is less than 100° F. Isopropanol is less a concern with respect to flammability, but with government regulations concerning volatile organic compounds (VOCs), its use in bactericidal formulations is problematic.

Phenols are widely used for bactericidal action. Highly efficacious, phenols work by precipitating structural and enzymatic proteins thus inactivating the cellular machinery and ultimately leading to cell death. Phenolics used in the formulation of mycobactericidal compositions include ortho phenyl phenol, paratertiary amyl phenol, and benzyl chlorophenol. Phenols have a strong characteristic odor and are quite toxic. Even recently developed phenols which have high molecular weights, have a pungent odor, and, although less toxic than phenol itself, their level of toxicity is still a concern. With increasing molecular weight comes decreasing solubility, and compounds such as paratertiary amylphenol are relatively insoluble in water.

Compositions containing iodophors have been used against mycobacteria. Iodophors have a pervasive iodine smell and will stain any surface with which they come in contact.

Quaternary ammonium salt formulations have been used as disinfectants for many years and these formulations have broad spectrum antimicrobial activity. Although formulations containing higher concentrations of quaternary ammonium salts are known to be effective against certain gram positive and gram negative bacteria, these formulations do not display any tuberculocidal activity.

It has been surprisingly discovered that flowable mixtures of anionic and anti-microbial quaternary ammonium compounds can be prepared without losing the intrinsic antimicrobial activity associated with the quaternary ammonium compound. The anti-microbial compositions and surfactant blends of the present invention are flowabl at concentrations as high as about 80 percent by weight. While not required, such compositions optionally contain organic solvents.

The surfactant blends of the present invention are useful for preparing a variety of finished consumer cleaning products, including for example, liquid dish detergents, laundry detergents, automatic dishwasher detergents, hand soaps, laundry bars, personal cleansing bars, multi-purpose cleaners, multi-functional shampoos, body washes, and textile treatment compositions. Surfactant blends of the present invention also may be employed as surfactants in agricultural and pesticide applications. Surfactant blends of the present invention may be prepared in various concentrations and exhibit a wide range of rheological behavior. The surfactant blends display excellent detergent and conditioning properties.

As discussed in more detail below, in one aspect, the surfactant blends of the invention are anti-microbial compositions.

One aspect of the present invention relates to surfactant complexes comprising at least one cationic surfactant, at least one anionic surfactant, and at least one “bridging surfactant” selected from nonionic, semi-polar nonionic, and amphoteric/zwitterionic surfactants, and mixtures thereof. These complexes are useful as rheology modifiers in a wide variety of surfactant compositions.

The present invention furnishes antimicrobial ternary surfactant blends or compositions. Such compositions display effective, even enhanced, antimicrobial activity. These blends may also be used effectively to remove greasy, oily soil from surfaces and textiles, while at the same time disinfecting or sanitizing those materials. Further, depending on the specific choice of components, the invention is also capable of providing conditioning properties to skin, hair and textiles.

Surprisingly, it has been discovered that complexes of anionic and cationic surfactants can be utilized in combination with a bridging surfactant to produce ternary surfactant blends which allow the anionic-cationic complex to remain relatively soluble in aqueous solutions, and at a variety of concentrations, without the use of solubilizing organic solvents or insertion of alkoxy chains into the anionic or cationic surfactants. Surprisingly, blends of the present invention generally are flowable at concentrations as high as about 80 percent by weight. Additionally, the surfactant blends when diluted to a concentration of about 0.1 percent by weight in water generally form a clear aqueous solution substantially free of precipitates. As used herein, the term “flowable” means fluid under gravity at ambient conditions (about 1 atmosphere of pressure at about 25° C.) without application of mechanical energy. As used herein, the term “clear” means allowing at least 50% transmittance measured spectrophotometrically at 700 nanometers using water as the standard for 100% transmittance. Typically, the ternary suriactant blend comprises (a) at least one cationic surfactant, (b) at least one anionic surfactant, and (c) at least one bridging surfactant, wherein the molar ratio of (a):(b):(c) is generally about 1:1:1. However, to optimize performance, the molar ratio of the components can vary as conditions may dictate.

In one aspect, the invention provides a surfactant blend comprising:

(a) an antimicrobial compound of the formula:

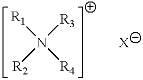

wherein

R₁ and R₂ are straight or branched chain lower alkyl groups having from one to seven carbon atoms;

R₃ is a straight or branched chain higher alkyl group having from about eight to twenty carbon atoms, or a benzyl group optionally substituted with C₁-C₆ alkyl;

R₄ is a straight or branched chain higher alkyl group having from about eight to twenty carbon atoms; and

X is an anion forming a water soluble salt, such as, halogen, methosulfate, saccharinate, sulfate, ethosulfate, tosylate, acetate, phosphate, nitrate, sulfonate, or carboxylate;

(b) an anionic surfactant which is

(i) an alkyl sulfate having an average of from about 8 to about 16 carbon atoms;

(ii) an alkyl sulfonate having an average of from about 8 to about 18 carbon atoms;

(iii) an alkyl ether sulfate having an average of from about 8 to about 16 carbon atoms in the alkyl portion and from about 1 to about 30 moles of ethylene oxide;

(iv) an a-olefin sulfonate having an average of from about 12 to about 18 carbon atoms;

(v) an α-sulfonat d C₁-C₆ alkyl ester of a fatty acid having an average of from about 11 to about 16 carbon atoms;

(vi) a sulfosuccinate having an average of from about 10 to about 16 carbon atoms;

(vii) a sarcosinate having an average of from about 10 to about 16 carbon atoms; or

(viii) a sulfoacetate having an average of from about 12 to about 20 carbon atoms;

or mixtures thereof;

(c) a bridging surfactant selected from the group consisting of amine oxides, ethoxamides, and betaines; and

optionally (d) a cationic surfactant which is a quaternary ammonium compound of the formula:

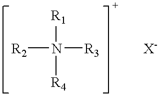

where

R₁, R₂, and R₃ are independently ethyl or methyl;

R₄ is an alkyl group having an average of from about 8 to about 16 carbon atoms; and

X is halogen, sulfate, methosulfate, ethosulfate, tosylate, acetate, phosphate, nitrate, sulfonate, or carboxylate;

wherein the total concentration of combined cationic, anionic, and bridging surfactants is from about 30 to about 80 percent by weight, and wherein the surfactant blend is flowable.

In another aspect, the invention provides a method of preparing a ternary surfactant composition comprising combining:

(a) an antimicrobial compound of the formula:

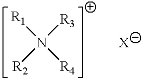

wherein

R₁ and R₂ are straight or branched chain lower alkyl groups having from one to seven carbon atoms;

R₃ is a straight or branched chain higher alkyl group having from about eight to twenty carbon atoms, or a benzyl group optionally substituted with C₁-C₆ alkyl;

R₄ is a straight or branched chain higher alkyl group having from about eight to twenty carbon atoms; and

X is an anion forming a water soluble salt, such as, halogen, methosulfate, saccharinate, sulfate, ethosulfate, tosylate, acetate, phosphate, nitrate, sulfonate, or carboxylate;

(b) an anionic surfactant which is

(i) an alkyl sulfate having an average of from about 8 to about 16 carbon atoms;

(ii) an alkyl sulfonate having an average of from about 8 to about 18 carbon atoms;

(iii) an alkyl ether sulfate having an average of from about 8 to about 16 carbon atoms in the alkyl portion and from about 1 to about 30 moles of ethylene oxide;

(iv) an α-olefin sulfonate having an average of from about 12 to about 18 carbon atoms;

(v) an α-sulfonated C₁-C₆ alkyl ester of a fatty acid having an average of from about 11 to about 16 carbon atoms;

(vi) a sulfosuccinate having an average of from about 10 to about 16 carbon atoms;

(vii) a sarcosinate having an average of from about 10 to about 16 carbon atoms; or

(viii) a sulfoacetate having an average of from about 12 to about 20 carbon atoms;

or mixtures thereof;

(c) a bridging surfactant selected from the group consisting of amine oxides, ethoxamides, and betaines; and

optionally (d) a cationic surfactant which is a quaternary ammonium compound of the formula:

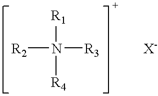

where

R₁, R₂, and R₃ are independently ethyl or methyl;

R₄ is an alkyl group having an average of from about 8 to about 16 carbon atoms; and

X is halogen, sulfate, methosulfate, ethosulfate, tosylate, acetate, phosphate, nitrate, sulfonate, or carboxylate;

wherein the bridging surfactant is added first or second, and wherein the total concentration of combined cationic, anionic, and bridging surfactants is from about 30 to about 80 percent by weight, and wherein the surfactant blend is flowable.

In still another aspect, the invention provides a method for enhancing the solubility of an anionic-cationic surfactant complex where the cationic surfactant is an anti-microbial quaternary ammonium compound, the method comprising combining:

(a) an anionic-cationic complex formed by combining in any order

(i) an antimicrobial compound of the formula:

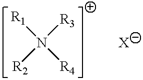

wherein

R₁ and R₂ are straight or branched chain lower alkyl groups having from one to seven carbon atoms;

R₃ is a straight or branched chain higher alkyl group having from about eight to twenty carbon atoms, or a benzyl group optionally substituted with C₁-C₆ alkyl;

R₄ is a straight or branched chain higher alkyl group having from about eight to twenty carbon atoms; and

X is an anion forming a water soluble salt, such as, halogen, methosulfate, saccharinate, sulfate, ethosulfate, tosylate, acetate, phosphate, nitrate, sulfonate, or carboxylate;

optionally (ii) a cationic surfactant which is a quaternary ammonium compound of the formula:

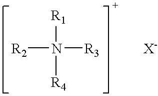

where

R₁, R₂, and R₃ are independently ethyl or methyl;

R₄ is an alkyl group having an average of from about 8 to about 16 carbon atoms; and

X is halogen, sulfate, methosulfate, ethosulfate, tosylate, acetate, phosphate, nitrate, sulfonate, or carboxylate; and

(ii) an anionic surfactant which is

(1) an alkyl sulfate having an average of from about 8 to about 16 carbon atoms;

(2) an alkyl sulfonate having an average of from about 8 to about 18 carbon atoms;

(3) an alkyl ether sulfate having an average of from about 8 to about 16 carbon atoms in the alkyl portion and from about 1 to about 30 moles of ethylene oxide;

(4) an α-olefin sulfonate having an average of from about 12 to about 18 carbon atoms;

(5) an α-sulfonated C₁-C₆ alkyl ester of a fatty acid having an average of from about 11 to about 16 carbon atoms;

(6) a sulfosuccinate having an average of from about 10 to about 16 carbon atoms;

(7) a sarcosinate having an average of from about 10 to about 16 carbon atoms; or

(8) a sulfoacetate having an average of from about 12 to about 20 carbon atoms;

or mixtures thereof; and

(b) a bridging surfactant selected from the group consisting of amine oxides, ethoxamides, and betaines.

In another aspect, the invention provide surfactant blends having antimicrobial properties. In this aspect, the blend compris s an anionic surfactant, a bridging surfactant, and a quaternary ammonium antimicrobial compound. Such blends synergistically enhance the anti-microbial efficiency of the antimicrobial compound. Such blends continue to display the excellent flowability associated with the individual surfactants employed in the anti-microbial blend as well as the other surfactant blends of the invention generally.

This invention further relates to personal care, laundry, and cleaning products, including for example, creams, lotions, sunscreens, hand soaps, laundry bars, personal cleansing bars, multi-purpose cleaners, multifunctional shampoos, body washes, liquid dish detergents, laundry detergents, automatic dishwasher detergents and textile treatment compositions which contain the inventive compositions. The invention further relates to agricultural and pesticide formulations which contain the inventive compositions. In a related aspect, the invention provides hard surface cleaners, spray disinfectants, and industrial sanitizing compositions. These compositions may be concentrates or dilute mixtures of the antimicrobial ternary surfactant blends of the invention.

The invention further provides surfactant blends exhibiting excellent detergent properties comprising a synergistic mixture of anionic and cationic surfactants that are generally flowable at concentrations as high as about 80 percent by weight, and, when diluted to a concentration of about 0.1 percent by weight in water, generally form clear aqueous solutions substantially free of precipitates.

The antimicrobial compositions of the present invention optionally contain from about 0.001 percent to about 20 percent of optional ingredients such as those described in detail below and those generally selected from the group comprising anti-dandruff agents, fragrance oils, perfumes, coloring agents, dyes, sequestering agents, preservatives, pearlescent/suspending agents, thickener, viscosity modifiers, pH adjusting agents, gelling agents, opacifying agents, foam stabilizing auxiliary surfactants, silicone oils, non-volatile/nonionic silicone conditioning agents, vitamins, protein, sunscreen agents and mixtures thereof.

These and other aspects and advantages, as well as the scope, nature, and utilization of the invention will become apparent to those skilled in the art from the following detailed description and claims.

### DETAILED DESCRIPTION OF THE INVENTION

Cationic and anionic surfactants form complexes that are generally insoluble because the charged heads (anionic or cationic) responsible for water solubility are neutralized during complexation. It is the formation of these complexes that is believed to be responsible for decreasing or minimizing the antimicrobial activity of antimicrobial compounds contained in compositions also having an anionic surfactant.

Surprisingly, it has been found that H the cationic surfactant and anionic surfactant are combined with a bridging surfactant to form a ternary blend, a system displaying desirable and effective antimicrobial activity is produced. In ternary surfactant blends of the invention, the use of additional hydrophilic groups (such as ethylene oxide groups or additional charge that remains un-neutralized during complexation) on the anionic or cationic surfactant is not necessary to produce a flowable complex. Suitable flowability is obtained if an appropriate bridging surfactant is utilized in combination with the anionic and cationic surfactant.

The present invention provides ternary blends of cationic, anionic and bridging surfactants wherein anionic/cationic complexes are formed. While not intending to be limited by a particular theory, it is believed that the quaternary ammonium agent (a cationic surfactant) and anionic surfactants typically form ion pair complexes in aqueous solutions. Typically, the previously known ion pairs formed between antimicrobial quaternary ammonium compounds are incompatible and separate into layers after mixing. Further, such previously known ion pairs generally have low solubility and precipitate as a solid salt at typical use concentrations. It is believed that this eliminates the antimicrobial activity which prevents the use of such compositions as disinfectants. On the other hand, surprisingly the blends and compositions of the invention, which comprise ion pairs formed by quaternary ammonium antimicrobial compounds and many anionic surfactants together with a bridging surfactant, retain the antimicrobial activity of the ammonium compound. Such blends and compositions are flowable at concentrations of active components of up to about 80% by weight. These characteristics allow greater flexibility in using the bulk compositions (i.e. the surfactant blends), to formulate, for example, hard surface cleaners, antimicrobial coatings, spray disinfectants, and industrial sanitizing compositions. As noted, the ternary surfactant blends of the invention are generally flowable at concentrations as high as about 80 percent by weight.

Long-term storage stability is often lacking in mixtures employing anionic-cationic complex mixtures due to the tendency of anionic and cationic surfactants in combination to produce precipitates in water. Typically, such compositions are not stable and separate into two phases on storage, rendering them aesthetically and functionally unacceptable. Surprisingly, ternary surfactant blends of this invention are generally provided in the form of a flowable composition that can be expected to be stored for long periods of time prior to sale or use. The formation of an anionic-cationic precipitate is avoided herein, and a lack of such a precipitate in the compositions of this invention is one of this invention's advantages.

In another embodiment, the invention provides methods for preparing ternary surfactant blends. The ternary blends of the invention are readily obtained by merely pre-mixing either the anionic or the cationic surfactant with the bridging surfactant, followed by mixing with the surfactant not pre-mixed.

In other embodiments, the present invention provides complexes useful as rheology modifiers. The present invention further encompasses consumer detergent, laundry and personal care products prepared using the surfactant blends and/or complexes detailed herein. The essential, as well as the optional, components of the present invention are described below.

As noted above, the invention provides anti-microbial surfactant blends comprising a bridging surfactant, an anionic surfactant, and anti-microbial quaternary ammonium compound. These blends synergistically enhance the activity of the anti-microbial compound. Accordingly, lower concentrations of the anti-microbial compound may be used to provide about the same anti-microbial activity. Further, because the amount of anti-microbial compound may be reduced, the composition may contain higher concentrations of other active components and optional ingredients required to provide improved cleaning, foaming, and other properties.

The anti-microbial blends of the invention may be prepared with any of the anionic surfactants described herein. A preferred group of surfactants includes alkyl sulfates, alkyl sulfonates, and alkyl ether sulfates.

Another preferred group of anionic surfactants for use in the anti-microbial blends includes α-olefin sulfonates and α-sulfonated alkyl esters of fatty acids. A third preferred group includes sulfosuccinates and sarcosinates. A fourth preferred group of anionic surfactants is the sulfoacetates.

Preferred anionic surfactants are alkyl sulfonates, alkyl ether sulfates, and alkyl sulfates. Particularly preferred anionic surfactants are the alkyl sulfonates. Most preferred are the alkyl sulfonates having from about 8 to 18 carbon atoms. Particularly preferred are alkyl sulfonates having from about 8 to 10 carbon atoms.

The bridging surfactants suitable for use in the anti-microbial compositions are any of the nonionic surfactants described herein. Preferably, the non-ionic surfactants are amine oxides and alkanolamides. Somewhat less preferably but still suitable for use in the invention are betaines. The alkanolamides that are useful include both mono- and alkanolamides. Particularly preferred bridging surfactants are amine oxides and, most preferably, amine oxides having from about 10 to 18 and most preferably about 10 to 16 carbon atoms in the alkyl chain.

The anti-microbial blends of the invention may be prepared to have a broad range of concentrations. Generally speaking, concentrated anti-microbial blends of the invention will have concentrations of active components of (anionic surfactant, bridging surfactant, and anti-microbial compound) from about 15 to 50 weight percent, although high concentrations can be readily achieved. More preferably, concentrated anti-microbial blends will have concentrations of from about 30 to 40 percent by weight, while most preferred anti-microbial compositions will have concentrations in the range of about 35 to 40 percent by weight.

The concentrated anti-microbial blends may be diluted as necessary to any of a range of suitable concentrations effective for killing and/or inhibiting the proliferation of microorganisms. Accordingly, in these anti-microbial blends, the quaternary ammonium compound is found in an amount effective to kill and/or prevent proliferation or growth of bacteria. In the concentrated blends, the amount of the antimicrobial compound ranges generally from about 5 to 25 percent by weight, the bridging surfactant ranges in concentration from about 5 to 25 percent by weight of the surfactant blend, while the anionic surfactant ranges in concentration from about 2 percent to the about 15 percent by weight of the composition. Preferred concentrated anti-microbial blends of the invention include from about 10 to about 20 percent of the anti-microbial compound, from about 7 to about 15 percent by weight of the bridging surfactant, and from about 5 to about 15 percent by weight of the anionic surfactant

As noted above, the concentrated surfactant blends may be diluted to suitable “use” concentrations. In the context of the anti-microbial blends, they are generally be diluted such that the concentration of the anti-microbial compound ranges from about 1 to about 10,000 ppm. More preferably, the anti-microbial blends should be diluted such that the concentration of the anti-microbial compound is from about 3 to about 3000 ppm. Even more preferably, the anti-microbial blends should be diluted such that they have from about 3 to about 500 ppm of the anti-microbial compound. It is understood that effective concentrations of antimicrobial quaternary ammonium compounds vary depending upon the target organism. Accordingly, particularly preferred antimicrobial compositions are those where the concentration of the antimicrobial compound in a diluted state is tailored to be an amount effective to kill the microorganism or to inhibit or control the growth or proliferation of the microorganism. Such concentrations may be relatively low concentrations, e.g., from about 1-25 ppm, or relatively high concentrations, e.g., from about 200-300 ppm. Of course, other concentrations outside of these ranges will also be useful.

The essential, as well as the optional, components of the present invention are further described in detail below.

### Antimicrobial Compounds

The quaternary ammonium antimicrobial compounds useful in the invention have the general formula:

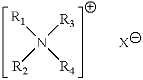

wherein R₁ and R₂ are straight or branched chain lower alkyl groups having from one to seven carbon atoms; R₃ is a straight or branched chain higher alkyl group having from about eight to twenty carbon atoms, or a benzyl group optionally substituted with C₁-C₆ alkyl; R₄ is a straight or branched chain higher alkyl group having from about eight to twenty carbon atoms; and X is a halogen or a methosulfate or saccharinate ion.

In preferred quaternary ammonium salts, R₁ and R₂ are methyl groups; R₃ is benzyl optionally substituted with C₁-C₆ alkyl, or straight or branched chain alkyl having from about eight to eighteen carbon atoms; and R₄ is straight or branched chain alkyl having from about eight to eighteen carbon atoms. A preferred X is halogen, more preferably, chlorine, or a methosulfate or saccharinate ion.

Illustrative of suitable quaternary ammonium microbiocides are: dioctyl dimethyl ammonium chloride, octyl decyl dimethyl ammonium chloride, didecyl dimethyl ammonium chloride, (C₁₂-C₁₈) n-alkyl dimethyl benzyl ammonium chloride, (C₁₂-C₁₈) n-alkyl dimethyl ethylbenzyl ammonium chloride, and (C₁₂-C₁₈) n-alkyl dimethyl benzyl ammonium saccharinate. This is not an exhaustive list and other quaternary ammonium salts having microbiocidal activity will suffice. The quaternary ammonium salt in the present invention need not be a single entity, but may be a blend of two or more quaternary ammonium salts. The amount, in weight-percent, of the quaternary ammonium salt, either as a single entity or blended, is typically from about 0.1%-2.0%. The preferred quatemary ammonium germicide is a mixture of about 34% by weight C₁₂ and 16% by weight C₁₄ n-alkyl dimethyl ethylbenzyl ammonium chloride and about 30% by weight C₁₄, 15% by weight C₁₆, 2.5% by weight C₁₂ and 2.5% by weight C₁₈ n-alkyl dimethyl benzyl ammonium chloride. Another prepared quatemary ammonium compound is n-alkyl(67% C₁₂, 25% C₁₄, 7% C₆, 1% C₈, C₁₀, C₁₈) dimethyl benzyl ammonium chloride. Another prepared anti-microbial compound is n-alkyl(50% C₁₂, 30% C₁₄, 17% C₁₆, 3% C₁₈) dimethyl benzyl ammonium chloride.

The following quatermary ammonium compounds may be efficiently used in the compositions of the invention. Each is commercially available from Stepan Company, Northfield, Ill. 60093.

| | |
|---|---|
| BTC ® 1010 | DIDECYL DIMETHYL AMMONIUM CHLORIDE |
| BTC ® 1010-80% | DIDECYL DIMETHYL AMMONIUM CHLORIDE |
| BTC ® 2125M | n-ALKYL DIMETHYL BENZYL AMMONIUM CHLORIDES (and) n-ALKYL |
| | DIMETHYL ETHYLBENZYL AMMONIUM CHLORIDES |
| BTC ® 2125M P40 | n-ALKYL DIMETHYL BENZYL AMMONIUM CHLORIDES (and) n-ALKYL |
| | DIMETHYL ETHYLBENZYL AMMONIUM CHLORIDES |
| BTC ® 2125M-80% | n-ALKYL DIMETHYL BENZYL AMMONIUM CHLORIDES (and) n-ALKYL |
| | DIMETHYL ETHYLBENZYL AMMONIUM CHLORIDES |
| BTC ® 2125M-90% | n-ALKYL DIMETHYL BENZYL AMMONIUM CHLORIDES (and) n-ALKYL |
| | DIMETHYL ETHYLBENZYL AMMONIUM CHLORIDES |
| BTC ® 2565 | n-ALKYL DIMETHYL BENZYL AMMONIUM CHLORIDE |
| BTC ® 2568 | n-ALKYL DIMETHYL BENZYL AMMONIUM CHLORIDE |
| BTC ® 50 NF | n-ALKYL DIMETHYL BENZYL AMMONIUM CHLORIDE |
| BTC ® 65 NF | n-ALKYL DIMETHYL BENZYL AMMONIUM CHLORIDE |
| BTC ® 776 | n-ALKYL DIMETHYL BENZYL AMMONIUM CHLORIDE and -DIALKYL |
| | METHYL BENZYL AMMONIUM CHLORIDE |
| BTC ® 818 | DIALKYL DIMETHYL AMMONIUM CHLORIDE |
| BTC ® 818-80% | DIALKYL DIMETHYL AMMONIUM CHLORIDE |
| BTC ® 824 | n-ALKYL DIMETHYL BENZYL AMMONIUM CHLORIDE |
| BTC ® 824 P100 | n-TETRADECYL DIMETHYL BENZYL AMMONIUM CHLORIDE |
| | MONOHYDRATE |
| BTC ® 8248 | n-ALKYL DIMETHYL BENZYL AMMONIUM CHLORIDE |
| BTC ® 8249 | n-ALKYL DIMETHYL BENZYL AMMONIUM CHLORIDE |
| BTC ® 835 | n-ALKYL DIMETHYL BENZYL AMMONIUM CHLORIDE |
| BTC ® 8358 | n-ALKYL DIMETHYL BENZYL AMMONIUM CHLORIDE |
| BTC ® 885 | n-ALKYL DIMETHYL BENZYL AMMONIUM CHLORIDE and DIALKYL |
| | DIMETHYL AMMONIUM CHLORIDE |
| BTC ® 888 | n-ALKYL DIMETHYL BENZYL AMMONIUM CHLORIDE and DIALKYL |
| | DIMETHYL AMMONIUM CHLORIDE |
| BTC ® 99 | DIDECYL DIMETHYL AMMONIUM CHLORIDE |

Preferred antimicrobial compounds of the invention include BTC 65NF, BTC 835, BTC 2125M, BTC 2125M P40, BTC 2125M-80%, and BTC 2125M-90%.

The antimicrobial compositions of the invention require an amount of the antimicrobial compound sufficient to provide a suitable concentration upon dilution to a final use volume such that there is an effective amount of the antimicrobial compound present in the dilute volume to control the growth of microorganisms. However, the antimicrobial composition may also contain other cationic components. Accordingly, the invention encompasses concentrated and dilute antimicrobial blends or compositions that include an anionic surfactant, a bridging surfactant, and an ammonium compound or mixtures thereof. Such mixtures would necessarily include at least one antimicrobial quaternary ammonium compound as described above optionally in combination with one or more additional cationic compounds. Such cationic compounds, termed “cationic surfactants” herein, may be added as deemed necessary to modify other characteristics of the composition.

### Auxiliary Cationic Surfactants

Although the antimicrobial surfactant is the principle cationic material present in the invention, the compositions may additionally comprise auxiliary cationic surfactants, used in combination with the antimicrobial surfactant. Generally, the auxiliary cationic surfactant is a surfactant selected from the group comprising fatty amine salts, fatty diamine salts, polyamine salts, quaternary ammonium salts, polyoxyethyleneated fatty amine salts, quaternized polyoxyethyleneated fatty amines, and mixtures thereof. A variety of cationic surfactants useful in the present invention are well known in the art Cationic surfactants useful herein include those disclosed in the following documents, all of which are incorporated by reference herein: M. C. Publishing Co., McCutcheon's Detergents & Emulsifiers, (North American Ed., 1993); Schwartz et al., Surface Active Agents, Their Chemistry and Technology, New York; Interscience Publisher, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr, issued Jun. 7, 1983. Suitable anions include but are not limited to halogen, sulfate, methosulfate, ethosulfate, tosylate, acetate, phosphate, nitrate, sulfonate, and carboxylate.

Auxiliary cationic surfactants in the form of quaternary ammonium salts include mono-long chain alkyl-tri-short chain alkyl ammonium halides, wherein the long chain alkyl group has from about 8 to about 22 carbon atoms and is derived from long-chain fatty acids, and wherein the short chain alkyl groups can be the same or different but preferably are independently methyl or ethyl.

Examples of auxiliary quaternary ammonium salts useful herein include but are not limited to cetyl trimethyl ammonium chloride and lauryl trimethyl ammonium chloride.

Auxiliary salts of primary, secondary and tertiary fatty amines are also suitable cationic surfactant materials. The alkyl groups of such amine salts preferably have from about 12 to about 22 carbon atoms, and may be substituted or unsubstituted. Secondary and tertiary amine salts are preferred, tertiary amine salts are particularly preferred. Suitable amine salts include the halogen (i.e fluoride, chloride, bromide), acetate, phosphate, nitrate, citrate, lactate and alkyl sulfate salts. Amine salts derived from amine, such as for example, stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (5 moles E.O.) stearylamine, dihydroxy ethyl stearylamine, and arachidylbehenylamine, are useful herein. Such salts also include stearylamine hydrogen chloride, soyamine chloride, stearylamine formate, N-tallowpropane diamine dichloride and stearamidopropyl dimethylamine citrate. Additionally cationic surfactants included among those useful in the present invention are disclosed in U.S. Pat. No. 4,275,055, Nachtigal, et al., issued Jun. 23, 1981, incorporated herein by reference.

In addition to the above, auxiliary cationic surfactants particularly useful herein are those of the general formula:

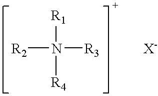

where R₁, R₂, and R₃ are independently ethyl or methyl; R₄ is an alkyl group having an average of from about 8 to about 16 carbon atoms; and X is an a suitable ion including but not limited to halogen, sulfate, methosulfate, ethosulfate, tosylate, acetate, phosphate, nitrate, sulfonate, or carboxylate.

Other auxiliary quaternary ammonium compounds and amine salt compounds include those of the above general formula in the form of ring structures formed by covalently linking two of the radicals. Examples include imidazolines, imidazoliniums, and pyridiniums, etc., wherein said compound has at least one nonionic hydrophile-containing radical as set forth above. Specific examples include 2-heptadecyl-4,5-dihydro-1H-imidazol-1-ethanol, 4,5-dihydro-1-(2-hydroxyethyl)-2-isoheptadecyl-1-phenylmethylimidazolium chloride, and 1-[2-oxo-2-[[2-[(1-oxoctadecyl)oxy]ethyl]amino]ethyl] pyridinium chloride. Additionally, useful polymerizable surface active agents include those of the above general formula in the form of ring structures formed by covalently linking two of the R₁-R₄ groups.

The auxiliary quaternary ammonium salts of the present invention may be prepared by a variety of methods known to the art, including for example, halide exchange, wherein a halide based quaternary ammonium compound is ion exchanged with X, where X is defined above.

Preferred auxiliary ationic surfactants for use in combination with the antimicrobial compounds in the compositions of the invention include octyltrimethyl ammonium chloride, decyltrimethyl ammonium chloride, dodecyltrimethyl ammonium bromide, dodecyltrimethyl ammonium chloride, and Cetac®-30, all of which are commercially available from Stepan Company.

Other auxiliary cationic surfactants includes those compounds commonly referred to as “ester quats”, and as disclosed in U.S. Pat. No. 5,939,059 (incorporated herein in its entirety). Typically, such materials are of the general formula

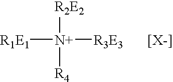

wherein X⁻ is an anion, R₁, R₂, R₃ can be the same or different and are independently straight or branched chain alkyl groups of from 2 to about 6 carbon atoms; R4 is a saturated or unsaturated, straight or branched chain aliphatic group having from about 1 to about 6 carbon atoms optionally substituted with hydroxy, epoxy or halogen and E₁, E₂ and E₃ can be the same or different at each occurrence and are selected from the group consisting of —H, —OH and aliphatic ester groups of the following formula (II):

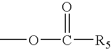

wherein R₅ is a saturated or unsaturated, straight or branched chain aliphatic group of from about 11 to about 24 carbon atoms, optionally substituted with hydroxy, epoxy or halogen, provided that at least one of E₁, E₂ or E₃ is a group of formula (II).

Additionally, an auxiliary quaternary ammonium compound of the formula:

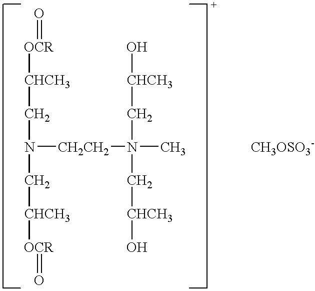

where R is substantially linear nor-oleyl, may be used in the various inventive blends. This material, also called STEPANQUAT™ ML, is commercially available from Stepan Company, Northfield, Ill. Additionally, the cationic surfactant may be a di-quaternary or poly quaternary compound. The auxiliary cationic surfactant may also be a DMAPA amidoamine based quaternary ammonium material.

### Anionic Surfactants

The anionic surfactants that may be utilized according to the present invention are well known to the art and are described below in a representative manner. Generally speaking, a variety of anionic surfactants useful in the present invention are well known in the art. Anionic surfactants useful herein include those disclosed in the following documents, all of which are incorporated by reference herein: M. C. Publishing Co., McCutcheon's Detergents & Emulsifiers, (North American Ed., 1993); Schwartz et al., Surface Active Agents, Their Chemistry and Technology, New York; Interscience Publisher, 1949; U.S. Pat. No. 4,285,841, Barrat et al, issued Aug. 25, 1981; and U.S. Pat. No. 3,919,678, Laughlin et al, issued Dec. 30, 1975.

Theanionic surfactants of the present invention generally include salts (including, for example, sodium, potassium, ammonium, and substituted ammonium salts such as mono-, di-, and triethanolamine salts) of the anionic sulfate, sulfonate, carboxylate and sarcosinate surfactants. Other suitable anionic surfactants include the isethionates such as the acyl isethionates, N-acyl taurates, fatty acid amides of methyl tauride, alkyl succinates, sulfoacetates, and sulfosuccinates, monoesters of sulfosuccinate (especially saturated and unsaturated C₁₂-C₁₈ monoesters), diesters of sulfosuccinate (especially saturated and unsaturated C₆-C₁₄ diesters), and N-acyl sarcosinates. Resin acids and hydrogenated resin acids are also suitable, such as rosin, hydrogenated rosin, and resin acids and hydrogenated resin acids present in or derived from tallow oil.

Anionic sulfate surfactants suitable for use in the compositions of the invention include the linear and branched primary and secondary alkyl sulfates, alkyl ethoxysulfates, fatty oleyl glycerol sulfates, alkyl phenol ethoxylate sulfates, alkyl phenol ethylene oxide ether sulfates, the C₅-C₁₇ acyl-N—(C₁-C₄ alkyl) and —N—(C₁-C₂ hydroxyalkyl)glucamine sulfates, and sulfates of alkylpolysaccharides such as the sulfates of alkylpolyglucoside.

Alkyl sulfate surfactants are preferably selected from the group consisting of the C₈-C₂₂ alkyl sulfates. Most preferably, the alkyl sulfate surfactant is a C₈-C₁₆ alkyl sulfate. Alkyl ethoxysulfate surfactants are preferably selected from the group consisting of the C₈-C₂₂ alkyl sulfates that have been ethoxylated with from about 0.5 to about 30 moles of ethylene oxide per molecule. Most preferably, the alkyl ethoxysulfate surfactant is a C₈-C₁₆ alkyl sulfate which has been ethoxylated with from about 1 to about 30 moles of ethylene oxide.

A particularly preferred anionic surfactant comprises mixtures of C₈ alkyl sulfate (POLYSTEP® B-29, commercially available from Stepan Company, Northfield, Ill.) and alkyl ethoxysulfate surfactants. Such mixtures have been disclosed in WO 93/18124, incorporated by reference herein.

Anionic sulfonate surfactants suitable for use herein include the salts of C₅-C₂₀ linear alkylbenzene sulfonates, alkyl ester sulfonates, C₆-C₂₂ primary or secondary alkane sulfonates, C₆-C₂₄ olefin sulfonates, alkyl glycerol sulfonates, fatty acyl glycerol sulfonates, fatty oleyl glycerol sulfonates, and any mixtures thereof.

Anionic sulfonate surfactants are preferably selected from the group consisting of the C₉-C₂₂ alkyl sulfonates and C₈-C₂₂ α-olefin sulfonates. Most preferably, the anionic sulfonate surfactant is an C₈-C₁₈ alkyl sulfonate, such as BIOTERGE® PAS-8S (commercially available from Stepan Company, Northfield, Ill.), or a C₁₂-C₁₈ α-olefin sulfonate, such as BIOTERGE® AS-40 (commercially available from Stepan Company, Northfield, Ill.).

Suitable anionic carboxylate surfactants include the alkyl ethoxy carboxylates, the alkyl polyethoxy polycarboxylate surfactants and the soaps (“alkyl carboxyls”), especially certain secondary soaps as described herein.

Suitable alkyl ethoxy carboxylates include those with the formula RO(CH₂CH₂O)ₓCH₂COO⁻M⁺ wherein R is a C₈ to C₁₈ alkyl group, x ranges from 0 to 10, and the ethoxylate distribution is such that, on a weight basis, the amount of material where x is 0 is less than about 20 percent and M is a cation. Suitable alkyl polyethoxy polycarboxylate surfactants include those having the formula RO(CHR₁CHR₂)R₃ wherein R is a C₆ to C₁₆ alkyl group, x ranges from 1 to 25, R₁ and R₂ are selected from the group consisting of hydrogen, methyl acid radical, succinic acid radical, hydroxysuccinic acid radical, and mixtures thereof, and R₃ is selected from the group consisting of hydrogen, substituted or unsubstituted hydrocarbon having between 1 and 8 carbon atoms, and mixtures thereof.

Suitable anionic soap surfactants include the secondary soap surfactants which contain a carboxyl unit connected to a secondary carbon. Preferred secondary soap surfactants for use herein are water-soluble members selected from the group consisting of the water-soluble salts of 2-methyl-1-undecanoic acid, 2-ethyl-1-decanoic acid, 2-propyl-1-nonanoic acid, 2-butyl-1-octanoic acid and 2-pentyl-1-heptanoic acid.

Suitable anionic sulfosuccinates include those having the formula

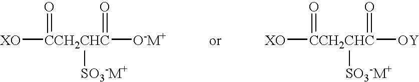

where

X and Y are the same or different and are selected from the group consisting of R and R(CH₂CH₂O), where x has an average value from about 1 to about 30;

R is C₈-C₂₂ alkyl;

and M is an counterion.

Anionic sulfosuccinate surfactants are preferably selected from the group consisting of the C₈-C₂₂ sulfosuccinates. Most preferably, the anionic sulfosuccinate surfactants is a mono-C₁₀-C₁₆ alkyl sulfosuccinate such as disodium laureth sulfosuccinate (STEPAN-MILD® SL3, commercially available from Stepan Company, Northfield, Ill.)

Other suitable anionic surfactants are the sarcosinates of the formula RCON(R₁)CH₂COOM, wherein R is a C₅-C₂₂ linear or branched alkyl or alkenyl group, R₁ is a C₁-C₄ alkyl group and M is an ion. Preferred sarcosinate surfactants include but are not limited to the myristyl and oleoyl methyl sarcosinates in the form of their sodium salts. Most preferably, the sarcosinate surfactant is a C₁₀-C₁₆ sarcosinate such as MAPROSYL® 30 (commercially available from Stepan Company, Northfield, Ill.).

Other suitable anionic surfactants are the alkyl sulfoacetates of the formula RO(CO)CH₂SO₃M, wherein R is a C₁₂-C₂₀ alkyl group and M is an ion. Preferred alkyl sulfoacetates include but are not limited to the lauryl and myristyl sulfoacetates in the form of their sodium salts. Most preferably, the alkyl sulfoacetate is LATHANOL® LAL (commercially available from Stepan Company, Northfield, Ill.).

The anionic surfactant may comprise

a) from about 3% to about 25% by weight based on the total weight of the composition of an alpha sulfonated alkyl ester of the formula

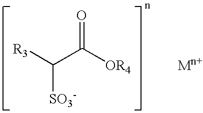

wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, or a mixture thereof; and

b) from about 0.01% to about 15% by weight based on the total weight of the composition of a sulfonated fatty acid of the formula

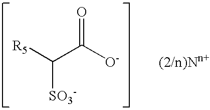

wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, or a mixture thereof; and wherein the weight ratio of i) to ii) is from about 10:1 to about 0.5:1. Additionally, the alkyl R₃ and/or R₅ groups can be a straight, branched, mid-chain branched or cyclic alkyl groups in form.

The alpha sulfonated alkyl esters used in the invention are typically prepared by sulfonating an alkyl ester of a fatty acid with a sulfonating agent such as S03, followed by neutralization with a base, such as sodium hydroxide, potassium hydroxide, calcium hydroxide, magnesium oxide, monoethanolamine, diethanolamine or triethanolamine, or a mixture thereof. When prepared in this manner, the alpha sulfonated alkyl esters normally contain a minor amount, typically not exceeding 33% by weight, of alpha sulfonated fatty acid, i.e., disalt, which results from hydrolysis of the ester. Generally, larger amounts of the disalt are obtained by hydrolyzing a known amount of the monosalt; hydrolysis may be accomplished in situ during the preparation of the composition. Accordingly, the alpha sulfonated alkyl ester and alpha sulfonated fatty acid may be provided to the composition (or utilized in the inventive process) as a blend of components which naturally result from the sulfonation of an alkyl ester of a fatty acid, or as individual components. Furthermore, it is known to one skilled in the art that minor impurities such as sodium sulfate, unsulfonated methyl esters (ME), and unsulfonated fatty acids (FA) may also be present in the mixtures according to the invention.

The alpha sulfonated alkyl esters, i.e., alkyl ester sulfonate surfactants, include linear esters of C₆-C₂₂ carboxylic acid (i.e., fatty acids) which are sulfonated with gaseous SO₃ according to the “The Journal of American Oil Chemists Society,” 52 (1975), pp. 323-329. Suitable starting materials include, among others, natural fatty substances as derived from tallow, palm oil, tc. Suitable anionic a-sulfonated methyl ester surfactant also include ALPHA STEP MG48 or ALPHA STEP ML-40 (both commercially available from Stepan Company, Northfield, Ill.).

The phthalate anionic surfactants useful herein have the general formula:

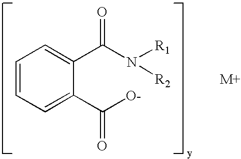

wherein R₁ and R₂ are independently selected from the group consisting essentially of H or C₁-C₄₀ linear or branched, substituted or unsubstituted alkyl, cycloalkyl, alkylene, alkaryl, aryl or R₃—O—R₄ groups, with R₃ and R₄ being independently selected from the group consisting essentially of C₁-C₂₂ linear or branched, substituted or unsubstituted alkyl, cycloalkyl, alkylene, alkaryl and aryl groups; y is an integer of a value satisfying the valency of M; and M is a cation and is preferably selected from the groups consisting of H, Na, K, NH₄ [including (CH₃CH₂)₃NH, (CH₃CH₂)₂NH₂, (HOCH₂CH₂)₃NH, (HOCH₂CH₂)₂NH₂ and similar ammonium derivatives], Ba, Ca, Mg, Al, Ti, Zr and mixtures thereof. These materials may be prepared as generally disclosed in WO 91/01970 (to Stepan Company), incorporated in its entirety.

Other anionic surfactant useful in the present invention N,N-disubstituted phthalamic acids and their salts as generally disclosed in U.S. Pat. Nos. 5,015,415 and 5,188,823 (both to Stepan Company), incorporated herein in their entirety. Phosphate esters are also generally useful anionic surfactants.

### Bridging Surfactants

The bridging surfactants of the present invention are selected from the group consisting of semi-polar nonionic (i.e., amine oxides), ethoxamide, and amphoteric surfactants (i.e.., betaines) and mixtures thereof. Especially preferred bridging surfactants include amine oxides, ethoxylated alkanolamides, and betaines.

Semi-polar nonionic surfactants include water-soluble amine oxides having an alkyl moiety containing from about 10 to about 18 carbon atoms and 2 moieties selected from the group consisting of alkyl groups and hydroxyalkyl groups containing from about 1 to about 3 carbon atoms. Semi-polar nonionic surfactants also include water-soluble sulfoxides having alkyl moieties containing from about 10 to about 18 carbon atoms and a moiety selected from the group comprising alkyl groups and hydroxyalkyl groups of from about 1 to about 3 carbon atoms.

The present invention encompasses semi-polar nonionic surfactants that are amine oxides formed as shown in Scheme 1. wherein R₁, R₂, R₃ independently are substituted or unsubstituted hydrocarbyl groups of from about 1 to about 30 carbon atoms, or hydrocarbyl groups having from about 1 to about 30 carbon atoms and containing one or more aromatic, ether, ester, amido, or amino moieties present as substituents or as linkages in the radical chain; and wherein X⁻ is an anion group selected from the group consisting of halogen, sulfonate, sulfate, sulfinate, sulfenate, phosphate, carboxylate, nitrate, and acetate. Additionally, useful semi-polar nonionic surfactants include those of the below general formula in the form of ring structures formed by covalently linking two of the R₁-R₄ groups. Examples include unsaturated imidazolines, imidazoliniums, and pyridiniums, and the like. Particularly preferred semi-polar nonionic surfactants include alkylamine and amidoamine oxides.

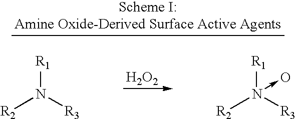

Particularly preferred amine oxides include but are not limited to AMMONYX® C8 (octylamine oxide), AMMONYX® C10 (decylamine oxide), AMMONYX® LO (laurylamine oxide), AMMONYX® MO (myristylamine oxide), AMMONYX® MCO (myristyl/cetylamine oxide), and AMMONYX® CDO (cocamidoproylamine oxide), all commercially available from Stepan Company, Northfield, Ill. Amine oxide surfactants which are generally suitable for use in the present invention are alkylamine and amidoamine oxides.

Other Examples of betaines and sultaines which are suitable for use in the present invention are alkyl betaines and sultaines sold as “Mirataine”® by Rhone Poulenc, “Lonzaine” by Lonza, Inc., Fairlawn, N.J. Examples of betaines and sultaines are cocobetaine, cocoamidoethyl betaine, cocoamidopropyl betaine, lauryl betaine, lauramidopropyl betaine, palmamidopropyl betaine, stearamidopropyl betaine, stearyl betaine, coco-sultaine, lauryl sultaine, tallowamidopropyl hydroxysultaine and the like.

Ethoxamides (also termed ethoxylated alkanolamides or polyethylene glycol amides) suitable for use in the present invention include those having the formula

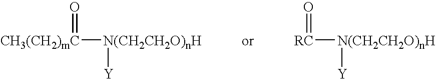

where

RCO—represents the fatty acids derived from coconut oil;

m is an integer from about 8 to about 16;

n has an average value of about 3;

Y is hydrogen or (CH₂CH₂O)pH; and

p is 0, 1 or more.

Preferred ethoxamides include but are not limited to AMIDOX® C-2 (PEG-3 cocamide), AMIDOX® C-5 (PEG-6 cocamide), and AMIDOX® L-5 (PEG-6 lauramide), all commercially available from Stepan Company, Northfield, Ill.

Suitable amphoteric surfactants are selected from the group consisting of alkyl glycinates, propionates, imidazolines, amphoacetates, amphoalkylsulfonates (sold under the tradename Miranol® by Rhone Poulenc), N-alkylaminopropionic acids, N-alkyliminodipropionic acids, imidazoline carboxylates, N-alkylbetaines, amido propyl betaines, sarcosinates, cocoamphocarboxyglycinates, amine oxides, sulfobetaines, sultaines and mixtures thereof. Additional suitable amphoteric surfactants include cocoamphoglycinate, cocoamphocarboxyglycinate, lauramphocarboxyglycinate, coco-amphopropionate, lauramphopropionate, stearamphoglycinate, cocoamphocarboxypropionate, tallowamphopropionate, tallowamphoglycinate, oleoamphoglycinate, caproamphoglycinate, caprylamphopropionate, caprylamphocarboxyglycinate, cocoyl imidazoline, lauryl imidazoline, stearyl imidazoline, behenyl imidazoline, behenylhydroxyethyl imidazoline, capryl-amphopropylsulfonate, cocamphopropylsulfonate, stearamphopropylsolfonate, oleoampho-propylsulfonate and the like.

Examples of betaines and sultaines which are suitable for use as bridging surfactants are alkyl betaines and sultaines sold under the tradename Mirataine by Rhone Poulenc, and Lonzaine by Lonza, Inc., Fairlawn, N.J. Additional examples of betaines and sultaines include cocobetaine, cocoamidoethyl betaine, cocoamidopropyl betaine, lauryl betaine, lauramidopropyl betaine, palmamidopropyl betaine, stearamidopropyl betaine, stearyl betaine, cocosultaine, lauryl sultaine, tallowamidopropyl hydroxysultaine and the like. Particularly preferred amphoteric surfactants include AMPHOSOL® CA (cocamidopropyl betaine) and AMPHOSOL® DM (lauryl betaine), both commercially available from Stepan Company, Northfield, Ill.

Other betaines useful in the present invention include compounds having the formula R(R₁)₂N⁺R₂COO⁻ wherein R is a C₆-C₁₈ hydrocarbyl group, preferably C₁₀-C₁₆ alkyl group, each r₁ is typically C₁-C₃, alkyl, preferably methyl, and R₂ is a C₁-C₅ hydrocarbyl group, preferably a C₁-C₅ alkylene group, more preferably a C₁-C₂ alkylene group. Examples of suitable betaines include coconut acylamidopropyldimethyl betaine; hexadecyl dimethyl betaine; C₁₂-C₁₄ acylamidopropylbetaine; C₈-C₁₄ acylamidohexyldiethyl betaine; 4-[C₁₄-C₁₆ acylmethylamidodiethylammonio]-1-carboxybutane; C₁₆-C₁₈ acylamidododimethylbetaine; C₁₂-C₁₆ acylamidopentanediethylbetaine; C₁₂-C₁₆ acylmethylamidodimethylbetaine. Preferred betaines are C₁₂-C₁₈ dimethylamoniohexanoate and the C₁₀-C₁₈ acylamidopropane (or ethane) dimethyl (or diethyl) betaines.

Other sultaines useful in the present invention include compounds having the formula R(R₁)₂N⁺R₂SO₃⁻, wherein R is a C₆-C₁₈ hydrocarbyl group, preferably a C₁₀-C₁₆ alkyl group, more preferably a C₁₂-C₁₃ alkyl group; each R₁ is typically C₁-C₃ alkyl, preferably methyl and R₂ is a C₁-C₆ hydrocabyl group, preferably a C₁-C₃ alkylene or, preferably, hydroxyalkylene group. Examples of suitable sultaines are C₁₂-C₁₄ dihydroxyethylammino propane sulfonate, and C₁₆-C₁₈ dimethylammonio hexane sulfonate, with C₁₂-C₁₄ amido propyl ammonio-2-hydroxypropyl sultaine being preferred.

### Optional Ingredients

The following optional ingredients can be present in various quantities. The ternary surfactant blends may be formulated with optional components, such as fragrances, emollient, solvents, humectants, optical brightners, thickeners, powders, viscosity modifiers, hydrotropes, preservatives, bluing agents, and dyes, to produce a wide variety of end use products.

Generally, a wide variety of functional materials can optionally be present in the invention compositions and/or finished formulations which contain the compositions. Non-limiting examples of such option functional materials, include absorbenst, anticaking agents, antioxidants, antiperspirants, anitsoils, soil antiredeposition agents, antistats, binders, carriers, chelating/sequestering agents, colorants, peralescents, conditioners, corrosion inhibitors, coupling agents, hydrotropes, defoamers, detergent builders, dispersants, emollients, enzymes, flocculants, florescent whitening agents, hair fixatives, humectants, lubricants, opacifiers, plasticizers, powders, preservatives, release agents, scale inhibitors, solubilizers, solvents, stablizers, suspending agents, thickeners, waxes and polishes, as generally disclosed in McCutcheon's Functional Materials, (1997 North American Edition).

Although the use of such optional components is not essential to the present invention, and may in fact be somewhat less preferred depending on the desired final formulation and end use application, suitable additional optional emollients useful in formulating with blends of the present invention include, for example, stearyl alcohol, glyceryl ricinoleate, glyceryl stearate, propane-1,2-diol, butane-1,3-diol, mink oil, cetyl alcohol, stearamidopropyl dimethylamine, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, di-n-butyl sebacate, isopropyl myristate, isopropyl palmitate, isopropyl stearate, butyl stearate, polyethylene glycol, triethylene glycol, lanolin, cocoa butter, butyl myristate, isostearic acid, palmitic acid, isopropyl linoleate, lauryl lactate, myristyl lactate, decyl oleate, and myristyl myristate, and mixtures thereof.

Although generally less preferred, optional solvents useful in formulating with blends of the present invention include, for example, ethyl alcohol, propylene glycol, water, isopropanol, castor oil, ethylene glycol monoethyl ether, diethylene glycol monobutyl ether, diethylene glycol monoethyl ether, dimethyl sulphoxide, dimethyl formamide, and tetrahydrofuran, and mixtures thereof.

Optional humectants useful in formulating with compositions of the present invention include, for example, glycerin, sorbitol, sodium 2-pyrrolidone-5-carboxylate, soluble collagen, dibutyl phthalate, propylen glycol, and gelatin, and mixtures thereof.

Optional swellable polymer thickening agents include, for example, methyl cellulose, ethyl cellulose, xanthan gum, gum arabic, gum karaya, guar gum, locust bean gum, ghatti gum, hydrolyzed starches, low molecular weight ethylene oxide polymers, low molecular weight propylene oxide polymers and mixtures thereof.

The optional pearlescent/suspending agents suitable for use in the present invention include any of several long chain acyl derivative materials or mixtures of such materials, such as long chain acyl derivatives, long chain amine oxides, and mixtures thereof, wherein such suspending/pearlescent agents are present in the composition in crystalline form. These pearlescent/suspending agents are described in U.S. Pat. No. 4,741,855, Grote and Russell, issued May 3, 1988, the disclosure of which is incorporated herein by reference in its entirety. Included are ethylene glycol esters of fatty acids having from about 16 to about 22 carbon atoms. Preferred are the ethylene glycol stearates, both mono- and distearate, but particularly the distearate containing less than about 7% of the monostearate. Other suspending agents found useful are alkanolamides, preferably with about 16 to about 18 carbon atoms. Preferred alkanolamides are stearic monoethanolamide, stearic diethanolamine, stearic monoisopropanolamide and stearic monoethanolamide stearate. Other long chain acyl derivatives include long chain esters of long chain fatty acids (e.g., stearyl stearate, cetyl palmitate, etc.); glyceryl esters (e.g., glyceryl distearate) and long chain ester of long chain alkanol amides (e.g., stearamide DEA distearate, stearamide MEA stearate).

Additional optional pearlescent/suspending agents suitable for use in the present invention are alkyl (C₁₈-C₂₂) dimethyl amine oxides, such as stearyl dimethyl amine oxide. If the compositions contain an amine oxide or a long chain acyl derivative as a surfactant the pearlescent/suspending function could also be provided by such surfactant and additional pearlescent/suspending agents may not be needed.

Further optional pearlescent/suspending agents that can be used are long chain acyl derivatives, including, for example, N,N-dihydroxycarbyl amido benzoic acid and soluble thereof (e.g., Na and K salts), particularly N,N-di(hydrogenated) C₁₆, C₁₈ and tallow amido benzoic acid species of this family, which are commercially available from Stepan Company (Northfield, Ill., USA).

Another type of pearlescenvsuspending agent which can be used in the present invention is xanthan gum. Xanthan gum is well known to those skilled in the art. For example, hair care compositions utilizing xanthan gum as a pearlescent/suspending agent for the silicone hair conditioning component are described in U.S. Pat. No.4,788,066, Bolich and Williams, issued Nov. 29, 1988, the disclosure of which is incorporated herein by reference in its ntirety. See also, Whistler, Roy L. Editor Industrial Gums—Polysaccharides and Their Derivatives, New York: Academic Press, 1973. Xanthan gum is commercially available from Kelco, a division of Merck & Co., Inc. as Keltrol.

Combinations of long chain acyl derivatives and xanthan gum are disclosed as pearlescent/suspending agents for silicone hair conditioners in U.S. Pat. No. 4,704,272, Oh et al., issued Nov. 3, 1987, both of which are incorporated herein by referenced in their entirety, and may also be used in the present compositions. Gel formulations have high levels of pearlescent/suspending agents relative to pourable, liquid formulations which used as the primary means of imparting gel-like viscosity. Optional gelling agents suitable for use in the present invention include, for example, hydroxy ethylcellulose.

Other optional conditioning agents include sucrogylericide materials, particularly those disclosed in U.S. Pat. No. 5,705,147, issued Jan. 6, 1998 to Stepan Company, incorporated herein in its entirety.

Optional powders useful in formulating with compositions of the present invention include, for example, chalk, talc, fullers earth, kaolin, starch, gums, colloidal silicon dioxide, sodium polyacrylate, tetra alkyl and/or trialkyl aryl ammonium smectites, chemically modified magnesium aluminum silicate, organically modified montmorillonite clay, hydrated aluminum silicate, fumed silica, carboxyvinyl polymer, cellulosics such as hydroxyethyl cellulose, and sodium carboxymethyl cellulose, ethylene glycol monostearate, zinc or magnesium stearate, zinc oxide and magnesium oxide, and mixtures thereof. These components may also be used as thickeners in fluid or semi-fluid compositions.

Examples of other optional ingredients useful in formulating with compositions of the present invention include, for example, silicone polymers; preservatives, such as para-hydroxy benzoate esters; humectants, such as butane-1,3-diol, glycerol, sorbitol, polyethylene glycol; stabilizers, such as sodium chloride or ammonium chloride; buffer systems, such as lactic acid together with a base such as sodium hydroxide; thickeners; activity enhancers; colorants; whiteners; fragrances; and bactericides, and mixtures thereof.

The inventive compositions may optionally include small amounts of oils which generally comprise one or more hydrophobic materials. Representative optional oils suitable for use in the inventive compositions include, but are not limited to silicon oil, mineral oil, a cosmetic ester or petrolatum, or a mixture thereof. Suitable cosmetic esters include for example, STEPAN™ IPM (isopropyl myristate) STEPAN™ IPP (isopropyl palmitate), STEPAN™ OCTYL PALMITATE (octyl palmitate), STEPAN™ OCTYL ISONONANOATE (octyl isononanoate), STEPAN™ ICS (isocetyl stearate). STEPAN™ BS (butyl stearate), STEPAN™ 653 (cetyl palmitate), STEPAN™ 654 (cetyl myristate), STEPAN™ CETYL ALCOHOL (cetyl alcohol), all commercially available from Stepan Company (Northfield, Ill.).

Other non-limiting examples of useful optional oils include crude petroleum oil, distilled petroleum oil₁ heavy paraffinic oil, asphaltene oil, linseed oil, tall oil, soybean oil alkyd, linseed oil alkyd, isopropyl palmitate (IPP)₁ isopropyl myristate, caprylic/capric triglyceride, lanolin, acetylated lanolin alcohol, dimethicone, hydrogenated vegetable oil, sesame oil, safflower oil, avocado oil, glycerine, propylene glycol, sorbitol, C₁₂-C₁₆ alcohol benzoates, cyclomethicone, dimethicone, cocoa butter, vitamin E acetate, squalane, sodium pyrolidone carboxylic acid, methyl glucose ether, panthenol, melanin, octyl isononanoate (OIN), octyl dodecyl neopentanoate (e.g. Elefac I-205), isohexadecane (e.g. Permethyl 101A), hydrogenated vegetable oil (e.g. Vegepure). Other suitable oils isopropyl myristate, triglycerides, and various silicones including dimethicones and cyclomethicones, etc. Additionally, hydrocarbon oils (e.g., pentane, hexane and C₇-C₂₂ hydrocarbons) are suitable for the instant invention.

Other optional oils include corn oil, cotton seed oil, tallow, lard, olive oil, palm kernel oil, rapeseed oil, safflower seed oil, sunflower seed oil, olive oil, sesame seed oil, coconut oil, arachis oil, castor oil and acetylated lanolin alcohols. Also useful are oils and waxes, such as Evening Primrose oil, beeswax, ozokerite wax, and paraffin wax.

Optional non-volatile, nonionic silicone materials suitable for the present invention are selected from the group comprising polyalkyl siloxanes, polyaryl siloxanes, polyalkylaryl siloxanes, polyether siloxane copolymers and mixtures thereof. The nonvolatile polyalkyl siloxane fluids that may be used include, for example, polydimethylsiloxanes, available, for example, from General Electric Company as SF 1075 methyl phenyl fluid or from Dow Coming as 556 Cosmetic Grade Fluid. The polyether siloxane copolymers that may be used include, for example, a polypropylene oxide modified dimethylpolysiloxane (E.g., Dow Coming DC-1248) although ethylene oxide or mixtures of ethylene oxide and propylene oxide may also be used.

Optional silicone fluids hereof also include polyalkyl or polyaryl siloxanes with the following general formula:

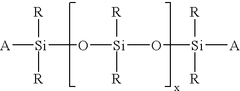

wherein R is alkyl or aryl, and x is an integer form about 7 to about 8,000. “A” represents groups which block the ends of the silicone chains. Suitable A groups include methyl, methoxy, ethoxy, propoxy, and aryloxy. The two R groups on the silicone atom may represent the same or different groups. Preferably, the two R groups represent the same group. Suitable R groups include, for example, methyl, ethyl, propyl, phenyl, methylphenyl and phenylmethyl. The somewhat preferred silicones are polydiemethyl siloxane, polydiethyl siloxane and polymethylphenyl siloxane. The silicone material for use in compositions of the invention can be for example, polyalkyl siloxanes, polyalkylaryl siloxanes, aminofunctional silicones, polydiorganosiloxanes or mixtures thereof may be used.

Optional silicone gums i.e., non volatile silicones, may be used as the silicone materials. For the purpose of the present invention, the term silicone gum denotes polydiorganosiloxanes having a molecular weight of 200,000 to 2,000,000. Examples of suitable optional silicone gums are for example described in U.S. Pat. No. 4,152,416. Specific examples of suitable silicone gums are polydimethyl or polydiphenyl siloxane polymers.

Such optional silicone materials for use in the compositions of the invention have a viscosity of 10⁴ to 10⁹ mpa.s at 25° C., more preferably from 5×10⁴ to 5×10⁸, most preferably from 10⁵ to 5×10⁷ mPa.s. A suitable method for measuring the viscosity is by means of a glass capillary viscometer (of Dow Corning CTM 0004), or by a Brookfields synchrolectric viscometer (of Dow Coming CTM 0050).

In certain embodiments of the invention, the optional oil also comprise a carrier or diluent material for the high viscosity, non-volatile silicone material. Often, high viscosity silicone materials are supplied as a dispersion in a carrier or diluent material, for example as a 5-25% by weight dispersion of the high viscosity silicone in cyclomethicone, linear dimethicone and/or isoparaffin. Alternatively or additionally the oil phase may comprise further diluents such as for example low viscosity silicones (having a viscosity of say between 0.1 to 1,000 mpa.s, more preferably o.5 to 500 mPa.s most preferably 0.65-100), liquid paraffins or methicones and other solvents such as C₁₀ to C₁₂ isoparaffins such as Isopar L (Esso), polyisobutene such as polysynlane (Nippon Oils and Fats), squalane such as Squalene (J. G. Marthens), branched chain hydrocarbons e.g., Permethyl 99A (Presperse), branched chain light paraffin oils such as Lytol (Witco) or WM1 (BP), mineral oil such as Marchol 82 (Esso) or Carnation Oil (Witco), long chain alkyl alkanoic esters such as decyl oleate (e.g., Cetiol V ex Henkel), isopropyl myristate (e.g., Estol 1514 ex Unichema) and glyceryl tri(2-ethyl hexanoate) e.g., Myritol CTEG ex Henkel).

The optional silicone oil may comprise a cyclomethicone or dimethicone. Generally such optional silicones may be represented by the formula:

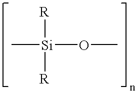

wherein R is a 1 to 3 carbon alkyl group, n is a number from 3 to 10, preferably from 3 to 7, and the unsatisfied valences on the oxygen and silicon atoms at the ends of the chain may be joined to one another to form a cyclic structure. Suitable optional volatile silicones are, for example, U.C.C. Y-7207, sold by Union Carbide Corporation in which each R is methyl and which typically comprises by weight 99.4% tetramer, 0.6% trimer and traces of the pentamer and hexamer; SWS-03314, sold by SWS Silicones, a Division of Stauffer Chemical Company, in which R is methyl and which is substantially all tetramer; and Dow Corning 344 fluid, sold by Dow Corning, Inc., in which R is methyl and which typically comprises by weight about 88% tetramer, about 11.8% pentamer and traces of trimer and hexamer.

The optional oils that may be used in the emulsions also include petroleum distillates, solvents and hydrocarbons such as, for example, mineral spirits, kerosene, terpenes, and glycol ethers.

Optional solid particulate matter useful in the instant invention includes solid materials listed below along with, for example, inorganic sunscreens, powders, pigments, abrasives, coal tar, antidandruff agents or a mixture thereof. Suitable anti-dandruff agents are zinc pyrithione, selenium sulfide, sulfur, zinc omadine, piroctone olamine and mixtures thereof.

The inventive compositions may also optionally include a single sunscreen or a mixture of more than one sunscreen. The optional sunscreens may be organic or inorganic sunscreens, or a combination of organic and inorganic sunscreens. Suitable optional sunscreens are those capable of blocking, scattering, absorbing or reflecting UV radiation. Inorganic sunscreens, often referred to as physical sunscreens, typically scatter, reflect and absorb UV radiation while organic sunscreens generally absorb UV radiation. Representative optional sunscreen components capable of protecting human skin from the harmful effects of UV-A and UV-B radiation are set forth below in Table A.

TABLE A

| CTFA Name | FDA Name/Chemical name |
|---|---|
| Benzophenone-3 | Oxybenzone/2-Hydroxy-4-methoxy |
| | benzophenone |
| Octylmethoxycinnamate | 2-Ethylhexyl-p-methoxy cinnamate |
| Benzophenone-4 | Sulisobenzone/2-Hydroxy-4-methoxy |
| | benzophenone-5-sulfonic acid |
| Octylsalicylate | 2-Ethylhexyl salicylate |
| Triethanolamine salicylate | Triethanolamine o-hydroxybenzoate |
| Glyceryl PABA | Glyceryl p-aminobenzoate |
| Padimate O | Octyldimethyl p-aminobenzoate |
| Homosalate | Homomenthyl salicylate |
| PABA | p-Aminobenzoic acid |
| Padimate A | Amyldimethyl PABA |
| Benzophenone-8 | Dioxybenzone |
| Octocrylene | 2-Ethyl-hexyl-2-cyano-3,3- |
| | diphenylacrylate |
| Phenyl Benzimidazole sulfonic | 2-Phenylbenzimidazole-5-sulfonic acid |
| acid | |
| Titanium dioxide | Titanium dioxide |
| Melanin coated titanium dioxide | |
| Zinc oxide | Zinc oxide |
| Avobenzone | Butyldibenzomethane |

Preferred optional sunscreens and sunscreen combinations are ethyl hexyl-p-methoxy-cinnamate (commercially available from Givaudan as Parsol MCX), Benzophenone-3 (Oxybenzone commercially available from Haarmann & Reimer), 2-phenylbenzimidazole-5-sulfonic acid (commercially available as Eusolex 232 from Rona), and octyldimethyl p-amino benzoic acid (octyl dimethyl PABA commercially available from Haarmann & Reimer).

Preferred optional inorganic (physical) sunscreens include appropriately sized particles of micronized titanium dioxide (TiO₂) and zinc oxide (ZnO). In addition, these particles may have various surface treatments to render the surface non-reactive and/or hydrophobic. Inorganic sunscreens may be added to the inventive formulations on a dry basis or as predispersed slurries.

In the case of predispersed slurries, well dispersed sluries are prefered. Representative non-limiting examples of currently preferred optional inorganic sunscreens include a slurry of 40% by weight of aluminum stearate coated micronized titanium dioxide in Octyl dodecylneopentanoate (commercially available as TiOSperse I from Collaborative Laboratories); a slurry containing 40% by weight of a mixture of TiO₂ and aluminum stearate in caprylic/capric triglyceride (commercially available as TiOSperse GT from Collaborative Laboratories); a 40% slurry of glycerol coated TiO₂ in butylene glycol and glycerin (commercially available as TiOSperse BUG/Gly from Collaborative Laboratories); melanin coated TiO₂ (commercially available from MelCo); ultrafine silicone coated TiO₂ (commercially available as UV-Titan from Presperse, Inc.); Dimethicone coated ZnO (commercially available as Z-cote HP1 from SunSmart, Inc.); a 60% TiO₂, aluminum stearate, an trifluoromethyl-C₁₋₄ alkyldimethicone in octyl dodecylneopentanoate (commercially available as ON60TA from Kobo Products, Inc.); and a 40% TiO₂ slurry in octyl palmitate (commercially available as Tioveil OP from Toxide Specialties, Ltd.).

Although not necessary and somewhat less preferred, composition of the instant invention can contain optional auxiliary emulsifiers. These optional emulsifiers typically include a low HLB materials such as glycerol esters including glycerol monostearate (GMS) and glycerol monooleate (GMO), ethylene glycol distearate (EGDS), PEG esters such as polyethylene glycol monostearate, polyglyceryl esters such as polyglyceryl-10-decaoleate (e.g.Drewpol), and silicone emulsifiers such as polysiloxane based water-in-oil emulsifiers (e.g. Abil EM-90). These auxiliary low HLB emulsifiers have HLB's of from about 1 to 6, and preferably from about 1.5 to about 3.8.

Although it is preferable that the inventive compositions (and methods to produce such compositions) are free of nonionic surfactants, the inventive compositions may optionally contain auxiliary nonionic surfactants. The auxiliary nonionic surfactants that may be utilized according to the present invention are well known to the art and are described below in a representative manner.

Suitable auxiliary nonionic surfactants in accordance with the present invention are generally disclosed at column, 13 line 14 through column 16, line 6 of U.S. Pat. No. 3,929,678, the disclosure of which is incorporated herein by reference in its entirety. Generally, the auxiliary nonionic surfactant is selected from the group comprising polyoxyethyleneated alkylphenols, polyoxyethyleneated straight chain alcohols, polyoxyethyleneated branched chain alcohols, polyoxyethyleneated polyoxypropylene glycols, polyoxyethyleneated mercaptans, fatty acid esters, glyceryl fatty acid esters, polyglyceryl fatty acid esters, propylene glycol esters, sorbitol esters, polyoxyethyleneated sorbitol esters, polyoxyethylene glycol esters, polyoxyethyleneated fatty acid esters, primary alkanolamides, ethoxylated primary alkanolamides, secondary alkanolamides, ethoxylated secondary alkanolamides, tertiary acetylenic glycols, polyoxyethyleneated silicones, N-alkylpyrrolidones, alkylpolyglycosides; alkylpolylsaccharides, EO-PO blockpolymers, polyhydroxy fatty acid amides, amine oxides and mixtures thereof. Further, exemplary, non-limiting classes of useful auxiliary nonionic surfactants are listed below:

1. The polyethylene, polypropylene, and polybutylene oxide condensates of alkyl phenols. In general, the polyethylene oxide condensates are preferred. These compounds include the condensation products of alkyl phenols having an alkyl group containing from about 6 to 12 carbon atoms in either a straight or branched chain configuration with the alkylene oxide. In a preferred embodiment, the ethylene oxide is present in an amount equal to from about 1 to about 25 moles of ethylene oxide per mole of alkyl phenol. Commercially available auxiliary nonionic surfactants of this type include Igepal® CO-630, marketed by the GAF Corporation; and Triton® X-45, X-114, X-100 and X-102, all marketed by the Rohm and Haas Company.

2. The condensation products of aliphatic alcohols with from about 1 to about 25 moles of ethylene oxide. The alkyl chain of the aliphatic alcohol can either be straight or branched, primary or secondary, and generally contain from about 8 to about 22 carbon atoms. Particularly preferred auxiliary nonionics are the condensation products of alcohols having an alkyl group containing from about 6 to about 11 carbon atoms with from about 2 to about 10 moles of ethylene oxide per mole of alcohol. Examples of commercially available auxiliary nonionic surfactants of this type include Tergitol® 15-S-9 (the condensation products of C₁₁-C₁₅ linear alcohol with 9 moles of ethylene oxide), Tergitol® 24-L-6 NMW (the condensation products of C₁₂-C₁₄ primary alcohol with 6 moles of ethylene oxide with a narrow molecular weight distribution), both marketed by Union Carbide Corporation; Neodol® 91-8 (the condensation product of C₉-C₁₁ linear alcohol with 8 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 91-6 (the condensation product of C₉-C₁₁ linear alcohol with 6 moles of ethylene oxide), marketed by Shell Chemical Company, and Kyro® EOB (the condensation product of C₁₃-C₁₅ linear alcohol with 9 moles of ethylene oxide), marketed by the Procter and Gamble Company.

3. The condensation products of ethylene oxide with a hydrophobic base formed by the condensation of propylene oxide with propylene glycol. The hydrophobic portion of these compounds preferably has a molecular weight of from about 1500 to about 1880 and exhibits water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially available Pluronic® surfactants, marketed by BASF.

4. The condensation products of ethylene oxide with the product resulting from the reaction of propylene oxide and ethylenediamine. The hydrophobic moiety of these products consists of the reaction product of ethylenediamine and excess propylene oxide, and generally has a molecular weight of from about 2500 to about 3000. This hydrophobic moiety is condensed with ethylene oxide to the extent that the condensation product contains from about 40% to about 80% by weight of polyoxyethylene and has a molecular weight of from about 5,000 to about 11,000. Examples of this type of nonionic surfactant include certain of the commercially available Tetronic® compounds, marketed by BASF.

5. Alkylpolysaccharides disclosed in U.S. Pat. No. 4,565,647, Lenado, issued Jan. 21, 1986, incorporated herein by reference, having a hydrophobic group containing from about 6 to about 30 carbon atoms, preferably from about 10 to about 16 carbon atoms and a polysaccharide, e.g., a polyglucoside, hydrophilic group containing from about 1.3 to about 10, preferably from about 1.3 to about 3, most preferably from about 1.3 to about 2.7 saccharide units. Any reducing saccharide containing 5 or 6 carbon atoms can be used, e.g., glucose, galactose and galactosyl moieties can be substituted for the glucosyl moieties. (Optionally, the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.

6. An ethyl ester ethoxylate and/or alkoxylate such as those described in U.S. Pat. No. 5,220,046, incorporated herein by reference. These material may be prepared according to the procedure set forth in Japanese Kokai patent application No. HEI 5 [1993]-222396. For example, they may be prepared by a one-step condensation reaction between an alkyl ester and an alkylene oxide in the present of a catalytic amount of magnesium together with another ion selected from the group of Al⁺³, Ga⁺³, In⁺³, Co⁺³, Sc⁺³, La⁺³ and Mn⁺³. Option desirably, there can be a polyalkyleneoxide chain joining the hydrophobic moiety and the polysaccharide moiety. The preferred alkyleneoxide is ethylene oxide. Typical hydrophobic groups include alkyl groups, either saturated or unsaturated, branched or unbranched, containing from about 8 to about 18, preferably from about 12 to about 14 carbon atoms; n is 2 or 3, preferably 2; t is from about 0 to about 10, preferably 0; and x is from about 1.3 to about 10, preferably from about 1.3 to 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glucosyl units can then be attached between their 1-position and the preceding glycosyl units 2-, 3-, 4- and/or 6-position, preferably predominately the 2-position.

7. Other auxiliary nonionic surfactants include alkoxylated mono- and diglycerides of fatty acids. Preferred are ethoxylated and/or propoxylated glycerides of fatty acids having from 6-40 carbon atoms. Suitable alkoxylated mono- and diglycerides of such acids are commercially available from Witco Corporation. Examples of such auxiliary nonionic surfactants include Varonic LI-63 (PEG-30 Glyceryl Cocoate, Witco), Varonic LI-67 (PEG-80 Glyceryl Cocoate, Witco), Varonic LI-67, 75% (PEG-80 Glyceryl Cocoate, Witco), Varonic LI-42 (PEG-20 Glyceryl Tallowate, Witco), Varonic LI-48 (PEG-80 Glyceryl Tallowate, Witco), and Varonic LI-420, 70% (PEG-200 Glyceryl Tallowate, Witco).

8. Additional auxiliary nonionic surfactants are alkoxylated alkyl esters of fatty acids. Preferred auxiliary alkoxylated alkyl esters are ethoxylated and/or propoxylated methyl esters of fatty acids having from 8-40 carbon atoms. Suitable auxiliary alkoxylated methyl esters of such acids are commercially available from Lion Corporation. Examples of such auxiliary nonionic surfactants include RCO₂(CH₂CH₂O)ₙCH₃ where R is C₁₂, and n is about 10.9 (commercially available from Lion Corporation, Japan, as LC-110M), and RCO₂(CH₂CH₂O)ₙCH₃ where R is C₁₂, and n is about 14.6 (commercially available from Lion Corporation, Japan, as LC-150M-92).

The compositions of the present invention may also be formulated into finished detergent formulations, in combination with optional detergent builder materials. Nearly any detergent builders known in the art can be formulated with the present blends. Examples of useful detergent builders are described in U.S. Pat. Nos. 4,321,165, (to Smith et al, issued Mar. 23,1982) and 5,565,145 (to Watson et al., issued Oct. 15, 1996), both incorporated herein by reference. Detergent builders can optionally be included in the compositions herein to assist in-controlling mineral hardness. Inorganic as well as organic builders can be used. Builders are typically used in fabric laundering compositions to assist in the removal of particulate soils. The level of builder can vary widely depending upon the end use of the composition and its desired physical form. When present in a final formulation, the compositions will typically comprise at least about 1% builder. Uquid formulations typically comprise from about 5% to about 50%, more typically about 5% to about 30%, by weight, of detergent builder. Granular finished formulations typically comprise from about 10% to about 80%, more typically from about 15% to about 50% by weight, of the detergent builder. Lower or higher levels of builder, however, also can be acceptable.

Enzymes and enzyme stabilizers can be formulated with compositions of the instant invention for a wide variety of fabric laundering purposes, including removal of protein-based, carbohydrate-based, or triglyceride-based stains, for example, and for fabric restoration. Examples of useful enzymes and enzyme stabilizers are described in U.S. Pat. No. 5,565,145 (to Watson et al., issued Oct. 15, 1996), incorporated herein by reference. Useful enzymes include, for example, proteases, amylases, lipases, and cellulases, as well as mixtures thereof. Other types of enzymes may also be included. They may be of any suitable origin, such as vegetable, animal, bacterial, fungal and yeast origin. However, a particular enzyme choice is governed by several factors such as pH-activity and/or stability optima, thermostability, stability versus active detergents, builders and so on. In this respect bacterial or fungal enzymes are preferred, such as bacterial amylases and proteases, and fungal cellulases.

Suitable examples of proteases are the subtilisins which are obtained from particular strains of B. subtilis and B. licheniforms. Another suitable protease is obtained from a strain of Bacillus, having maximum activity throughout the pH range of 8-12, developed and sold by Novo Industries A/S under the registered trade name ESPERASE. The preparation of this enzyme and analogous enzymes is described in British Pat. Specification No. 1,243,784 of Novo. Proteolytic enzymes suitable for removing protein-based stains that are commercially available include those sold under the tradenames ALCALASE and SAVINASE by Novo Industries A/S (Denmark) and MAXATASE by International-Bio-Synthetics, Inc. (The Netherlands). Other proteases include Protease A (see European Patent Application 130,756, published Jan. 9, 1985) and Protease B (see European Patent Application Ser. No. 87303761.8, filed Apr. 28, 1987, and European Patent Application 130,756, Bott et al, published Jan. 9, 1985).

Amylases include, for example, amylases described in British Patent Specification No. 1,296,839 (Novo), RAPIDASE, International Bio-Synthetics, Inc. and TERMAMYL, Novo Industries.

Cellulases suitable for use with compositions of the present invention include both bacterial or fungal cellulase. Preferably, they will have a pH optimum of between 5 and 9.5. Suitable cellulases are disclosed in U.S. Pat. 4,435,307, Barbesgoard et al, issued Mar. 6, 1984, which discloses fungal cellulase produced from Humicola insolens and Humicola strain DSM1800 or a cellulase 212-producing fungus belonging to the genus Aeromonas, and cellulase extracted from the hepatopancreas of a marine mollusk (Dolabella Auricula Solander). Suitable cellulases are also disclosed in GB-A-2.075.028; GB-A-2.095.275 and DE-OS.247.832CAREZYME (Novo) is especially useful.

Suitable lipase enzymes include those produced by microorganisms of the Pseudomonas group, such as Pseudomonas stutzeri ATCC 19.154, as disclosed in British Patent. 1,372,034. See also lipases in Japanese Patent Application 53,20487, laid open to public inspection on Feb. 24, 1978. This lipase is available from Amano Pharmaceutical Co. Ltd., Nagoya, Japan, under the trade name Lipase P Amano, hereinafter referred to as Amano-P. Other commercial lipases include Amano-CES, lipases ex Chromobacter viscosum, e.g. Chromobacter viscosum var. lipolyticum NRRLB 3673, commercially available from Toyo Jozo Co., Tagata, Japan; and further Chromobacter viscosum lipases from U.S. Biochemical Corp., U.S.A. and Diosynth Co., The Netherlands, and lipases ex Pseudomonas gladioli. The LIPOLASE enzyme derived from Humicola lanuginosa and commercially available from Novo (see also EPO 341,947) is a preferred lipase for use herein.

A wide range of enzyme materials and means for their incorporation into synthetic detergent compositions are also disclosed in U.S. Pat. No. 3,553,139, issued Jan. 5, 1971 to McCarty et al. Enzymes are further disclosed in U.S. Pat. No. 4,101,457, Place et al, issued Jul. 18, 1978, and in U.S. Pat. No. 4,507,219, Hughes, issued Mar. 26, 1985, both. Enzyme materials useful for liquid detergent formulations, and their incorporation into such formulations, are disclosed in U.S. Pat. No. 4,261,868, Hora et al, issued Apr. 14, 1981. Enzymes for use in detergents can be stabilized by various techniques. Enzyme stabilization techniques are disclosed and exemplified in U.S. Pat. No. 3,600,319, issued Aug. 17, 1971 to Gedge, et al, and European Patent Application Publication No. 0 199 405, Application No. 20 86200586.5, published Oct. 29, 1986, Venegas. Enzyme stabilization systems are also described, for example, in U.S. Pat. No. 3,519,570.

The optional enzymes useful herein may be stabilized by the presence of water-soluble sources of calcium and/or magnesium ions in the finished compositions which provide such ions to the enzymes. Calcium ions are generally somewhat more effective than magnesium ions and are preferred herein if only one type of cation is being used. Additional stability can be provided by the presence of various other disclosed stabilizers, especially borate species. See Severson, U.S. Pat. No. 4,537,706. Typical detergents, especially liquids, will comprise from about 1 to about 30, preferably from about 2 to about 20, more preferably from about 5 to about 15, and most preferably from about 8 to about 12, millimoles of calcium ion per liter of finished composition. This concentration can vary somewhat, depending on the amount of enzyme present and its response to the calcium or magnesium ions. The level of calcium or magnesium ions should be selected so that there is always some minimum level available for the enzyme, after allowing for complexation with builders, fatty acids, etc., in the final composition. Any water-soluble calcium or magnesium salt can be used as the source of calcium or magnesium ions, including, but not limited to, calcium chloride, calcium sulfate, calcium maleate, calcium maleate, calcium hydroxide, calcium formate, and calcium acetate, and the corresponding magnesium salts. A small amount of calcium ion, generally from about 0.05 to about 0.4 millimoles per liter, is often also present in the final composition due to calcium in the enzyme slurry and formula water. In solid detergent compositions the final formulation may include a sufficient quantity of a water-soluble calcium ion source to provide such amounts in the laundry liquor. In the alternative, natural water hardness may suffice.

Generally, the aforementioned levels of calcium and/or magnesium ions are sufficient to provide enzyme stability to a finished formulation. More calcium and/or magnesium ions can be added to the compositions to provide an additional measure of grease removal performance. Accordingly, final formulations prepared from the blends disclosed herein typically will comprise from about 0.05% to about 2% by weight of a water-soluble source of calcium or magnesium ions, or both. The amount of water-soluble ion can vary with the amount and type of enzyme employed in the final composition.

Final compositions detailed herein, when utilized in a finished formulation, may also optionally contain various additional stabilizers, especially borate-type stabilizers. Boric acid is preferred, although other compounds such as boric oxide, borax and other borates (e.g., sodium ortho-, meta- and pyroborate, and sodium pentaborate) are suitable. Substituted boric acids (e.g., phenylboronic acid, butane boronic acid, and p-bromo phenylboronic acid) can also be used in place of boric acid.

Bleaching agents, bleach activators, chelating agents, anti-redeposition agents, polymeric dispersing agents, optical brighteners, suds suppressors, dye transfer inhibition agents, optical brighteners, and soil release agents can be formulated with blends of the instant invention. Examples of such materials are generally described in U.S. Pat. No. 5,565,145 (to Watson et al., issued Oct. 15, 1996), incorporated herein by reference.

Various other detergent additives or adjuvants may be present in the detergent product to give it additional desired properties, either of functional or aesthetic nature. Thus, there may be included in the formulation minor amounts of soil suspending or anti-redeposition agents, e.g. polyvinyl alcohol, fatty amides, sodium carboxymethyl cellulose, hydroxy-propyl methyl cellulose; optical brighteners, e.g. cotton, amine and polyester brighteners, for example, stilbene, triazole and benzidine sulfone compositions, especially, sulfonated substituted triazinyl stilbene, sulfonated naphthotriazole stilbene, benzidine sulfone, etc., most preferred are stilbene and triazole combinations.

Bluing agents such as ultramarine blue; enzymes, preferably proteolytic enzymes, such as subtilisin, bromelin, papain, trypsin and pepsin, as well as amylase type enzymes; bactericides, e.g. tetrachlorosalicylanilide, hexachlorophene; fungicides; dyes; pigments (water dispersible); preservatives; ultraviolet absorbers; anti-yellowing agents, such as sodium carboxymethyl cellulose, complex of C₁₂ to C₂₂ alkyl alcohol with C₁₂ to C₁₈ alkylsulfate; pH modifiers and pH buffers; color safe bleaches, perfume, and anti-foam agents or suds suppressors, e.g. silicon compounds, can also be used.

In the case of final formulations, other optional ingredients include neutralizing agents, buffering agents, phase regulants, hydrotropes, polyacids, suds regulants, opacifiers, antioxidants, preservatives, bactericides, dyes, perfumes, and brighteners described in the U.S. Pat. No. 4,285,841, Barrat et al, issued Aug. 25, 1981, incorporated herein by reference. Other ingredients useful in final detergent compositions can be formulated with blends of the instant invention, including carders, processing aids, pigments, solvents for liquid formulations, solid fillers for bar compositions, sodium sulfate, sodium chloride, protein hydrolysates, cholesterol derivatives, UV absorbers, chelating agents, etc. If high sudsing is desired, suds boosters such as the C₁₀-C₁₆ alkanolamides can be incorporated into the final compositions, typically at 1%-10% levels. The C₁₀-C₁₄ monoethanol and diethanol amides illustrate a typical class of such suds boosters. If desired, soluble magnesium salts such as MgCl₂, MgSO₄, and the like, can be added at levels of, typically, 0.1%-2%, to provide additional suds and to enhance grease removal performance to a final formulation.

Additionally, the compositions may optionally contain non-conventional surfactants, such as fluorosurfactants, gemini surfactants and polymeric cationic and polymeric anionic surfactants. The emulsification systems of the invention also comprise polysiloxane polyalkyl polyether copolymers, i.e., silicone glycol surfactants which are also known as copolyols. The amount of silicone glycol surfactant is preferably about 0.5 to 15% by weight. A more preferable amount of the silicone glycol surfactant is about 0.5-5% of the composition.

Suitable silicone surfactants are for example high molecular weight polymers of dimethyl polysiloxane with polyoxyethylene and/or polyoxypropylene side chains, having a molecular weight of from 10,000 to 50,000 and having the structure:

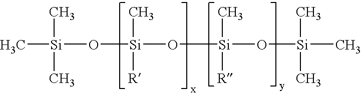

wherein the groups R′ are each chosen from —H, C₁₋₁₈ alkyl and R″ is —[CH₂CH₂O]ₐ[CH₂(CH₂)CHO]_{b}H, in which a has a value of from 9 to 115, b has a value of from 0 to 50, x has a value of from 133 to 673, y has a value of from 25 to 0.25. Preferably, the polymer is an alkoxylated polydimethyl polymer in which: a has a value of from 10 to 114, b has a value of from 0 to 49, x has a value of from 388 to 402, y has a value of from 15 to 0.75, Mthe group R″ having a molecular weight of from 1000 to 5000. A more preferred alkoxylated dimethyl polysiloxane polymer is one in which: a has the value 14, b has the value 13, x has the value 249, y has the value 1.25. A particularly preferred copolyol is cetyl dimethicone copolyol, available from T.H. Goldschmidt as Abil® EM-90.

Blends of the present invention are prepared from readily available, economical raw materials, and generally their preparation does not require any special handling or equipment. The blends may be prepared in a batch mode or a continuous mode.

Suitable preservatives are selected from the group comprising benzyl alcohol, methyl paraben, propyl paraben and imidazolidinyl urea.

Suitable thickeners and viscosity modifiers are selected from the group comprising diethanolamides of long chain fatty acids (e.g., PEG-3 lauramide), block polymers of ethylene oxide and propylene oxide such as Pluronic® F88 offered by BASF, Wyandotte, sodium chloride, sodium sulfate, ammonium xylene sulfonate, ethyl alcohol and polyhydridic alcohols such as, for example, propylene glycol and polyvinyl alcohol.

Suitable gelling agents include, for example, hydroxyethyl cellulose.

Suitable pH adjusting agents are selected from the group comprising citric acid, succinic acid, phosphoric acid, sodium hydroxide, sodiumcarbonate, etc.

Suitable sequestering agents include, for example, disodium ethylenediamine tetraacetate.

The compositions of the present invention typically contain water as the solvent; however, other solvents may optionally be employed, either alone or in combination with water. Low molecular weight primary or secondary alcohols, exemplified by methanol, ethanol, propanol, and isopropanol, are suitable optional solvents. Monohydric alcohols are preferred optional solvents, but polyols such as those containing from 2 to about 6 carbon atoms and from 2 to about 6 hydroxy groups (e.g., 1,3-propanediol, ethylene glycol, glycerine, and 1,2-propanediol) can also be used. The compositions may contain from about 5 to about 90 percent, typically from about 10 to about 50 percent by weight of water and/or optional solvent.

While pH is of secondary significance herein, the compositions of the present-invention typically are prepared having a pH of between about 2 and about 10, preferably between about 5 and about 8. Techniques for controlling pH at recommended usage levels include the use of buffers, alkalis, acids, etc., and are well known to those skilled in the art. Suitable materials for adjusting the pH of these compositions include triethanolamine, diethanolamine, sodium carbonate, sodium bicarbonate, and the like.

The ternary surfactant blends of the invention may be formulated into a variety commercially useful products. Additionally, the ternary surfactant blends may be processed into a variety of forms such as, for example, liquids, solutions, solids, powders, flakes, semi-solids, gels, “ringing” gels, G-phase liquids/pastes, hexagonal liquid crystal phases, or thick non-flowable pastes. The ternary surfactant blends may be spray dried, flaked, or extruded. Although not critical to the present invention, the blends may be prepared “neat” or in a conventional solvent such as water, low molecular weight alcohol or hydrocarbon, or a mixture thereof, to produce a solution of the ternary surfactant blend. The present invention encompasses ternary surfactant systems in dry form and as aqueous solutions. Ternary surfactant blends in concentrations up to 100 percent by weight may be isolated by drying a solution of the blend. Conversely, ternary surfactant blend solutions may be prepared by dissolving a solid form of the blend in water, low molecular weight alcohol, low molecular weight glycol, or mixtures thereof.

One skilled in the art will recognize that modifications may be made in the present invention without deviating from the spirit or scope of the invention. The invention is illustrated further by the following examples, which are not to be construed as limiting the invention or scope of the specific procedures or compositions described herein. All documents, e.g., patents and journal articles, cited above or below are hereby incorporated by reference in their entirety.

As used in the Examples appearing below, the following designations, symbols, terms and abbreviations have the indicated meanings:

| Material | Definition |
|---|---|
| Alpha Step ® MC-48 | Sodium alphasulfo methyl C₁₂₋₁₈ ester (and) disodium alphasulfo C₁₂₋₁₈ |
| | fatty acid salt (commercially available from Stepan Company, Northfield |
| | Illinois) |
| Alpha Step ® ML-40 | Sodium alphasulfo methyl ester (and) disodium alphasulfo lauric acid salt |
| | (commercially available from Stepan Company, Northfield Illinois) |
| BTC ® 2125M | n-Alkyl dimethyl benzyl ammonium chlorides (and) n-alkyl dimethyl |
| | ethylbenzyl ammonium chlorides (commercially available from Stepan |
| | Company, Northfield Illinois) |
| BTC ® 65 NF | n-Alkyl dimethyl benzyl ammonium chloride (commercially available from |
| | Stepan Company, Northfield Illinois) |
| Polystep ® B-29 | Sodium octyl sulfate (commercially available from Stepan Company, |
| | Northfield Illinois) |
| Polystep ® B-25 | Sodium decyl sulfate (commercially available from Stepan Company, |
| | Northfield Illinois) |
| Polystep ® B-22 | Ammonium lauryl ether sulfate (3EO) (commercially available from |
| | Stepan Company, Northfield Illinois) |
| Polystep ® B-20 | Ammonium lauryl ether sulfate (12EO) (commercially available from |
| | Stepan Company, Northfield Illinois) |
| Bioterge ® PAS-8S | Sodium octyl sulfonate (commercially available from Stepan Company, |
| | Northfield Illinois) |
| Maprosyl ® 30 | Sodium lauroyl sarcosinate (commercially available from Stepan |
| | Company, Northfield Illinois) |
| Stepan-Mild ® SL3 | Disodium laureth sulfosuccinate (commercially available from Stepan |
| | Company, Northfield Illinois) |
| Steol CS-370 | Sodium laureth sulfate (3EO) (commercially available from Stepan |
| | Company, Northfield Illinois) |
| Steol CS-460 | Ammonium laureth sulfate (3EO) (commercially available from Stepan |
| | Company, Northfield Illinois) |
| Stepanol ® WA-Extra | Sodium lauryl sulfate (commercially available from Stepan Company, |
| | Northfield Illinois) |
| Bioterge ® AS-40 | Sodium C14-16 olefin sulfonate (commercially available from Stepan |
| | Company, Northfield Illinois) |
| QC8 | Octyltrimethylammonium chloride |
| QC10 | Decyltrimethylammonium chloride |
| QC12 or DTMAB | Dodecyltrimethylammonium bromide |
| Cetac ® 30 | Cetyltrimethylammonium chloride (commercially available from Stepan |
| | Company, Northfield Illinois) |
| Ammonyx ® LO | Lauramine oxide (commercially available from Stepan Company, |
| | Northfield Illinois) |
| Ammonyx ® MCO | Myristyl/cetyl amine oxide (commercially available from Stepan Company, |
| | Northfield Illinois) |
| Ammonyx ® C8 | Octylamine oxide (commercially available from Stepan Company, |
| | Northfield Illinois) |
| Ammonyx ® C10 | Decylamine oxide (commercially available from Stepan Company, |
| | Northfield Illinois) |
| Amphosol ® CA | Cocamidopropyl betaine (commercially available from Stepan Company, |
| | Northfield Illinois) |
| Amphosol ® DM | Lauryl betaine (commercially available from Stepan Company, Northfield |
| | Illinois) |
| Amidox ® C-2 | PEG-3 Cocamide (commercially available from Stepan Company, |
| | Northfield Illinois) |
| Amidox ® C-5 | PEG-6 Cocamide (commercially available from Stepan Company, |
| | Northfield Illinois) |
| Amidox ® L-5 | PEG-6 Lauramide (commercially available from Stepan Company, |
| | Northfield Illinois) |

One skilled in the art will recognize that modifications may be made in the present invention without deviating from the spirit or scope of the invention. The invention is illustrated further by the following examples, which are not to be construed as limiting the invention or scope of the specific procedures or compositions described herein.

In the following examples, all amounts are stated in percent by weight of active material unless indicated otherwise. Surface tension measurements, Draves wetting measurements, and Ross Miles foaming initial and final measurements all were taken at 0.1% concentration of the surfactant blend in water.

### EXAMPLE 1

Various surfactant blends were prepared as shown in Table I. Each blend is prepared at an approximately 1:1:1 molar ratio. The appearance of each blend as a concentrated (30-40%) and dilute (0.1-1.0%) composition was noted.

None of the prepared blends comprising only anionic and cationic surfactants were single-phase at both concentrations. This observation confirms the limited solubility nature of anionic-cationic complexes.

Surprisingly, however, the addition of an amine oxide, ethoxamide, and/or betaine bridging surfactant to the anionic-cationic blends eliminated precipitation and produced clarity improvements in all cases, in most cases producing solutions that were clear at both concentrations. This observation demonstrates that the bridging surfactant promotes solubility of the anionic-cationic complex.

TABLE I

| | Appearance |
|---|---|
| Surfactant System | (concentrated) |
| Stepanol ® WA-Extra/QC8 | ppt |
| Stepanol ® WA-Extra/QC8/Amphosol ® CA | Clear |
| Stepanol ® WA-Extra/QC8/Ammonyx ® C10 | Clear |
| Polystep ® B-25/QC10 | ppt |
| Polystep ® B-25/QC10/Amphosol ® CA | No ppt —opaque |
| Polystep ® B-25/QC10/Ammonyx ® MCO | No ppt —opaque |
| Polystep ® B-29/QC10 | ppt |
| Polystep ® B-29/QC10/Amphosol ® CA | Clear |
| Polystep ® B-29/QC10/Ammonyx ® LO | Clear |
| Polystep ® B-29/QC10/Amidox ® C-5 | Clear |
| Steol ® CS-460/QC12 | ppt |
| Steol ® CS-460/QC12/Ammonyx ® LO | Clear |
| Steol ® CS-460/Cetac 30 | ppt |
| Steol ® CS-460/Cetac 30/Ammonyx ® LO/ | Clear |
| Alpha Step ® MC-48 | |
| PAS-8s/BTC 65NF | 2 layers |
| PAS-8s/BTC 65NF/Ammonyx LO | clear, flowable |
| CS 460/BTC 65NF | 2 layers |
| CS 460/BTC 65NF/Ammonyx LO | clear, flowable |

### EXAMPLE 2

A ternary surfactant blend of an anionic surfactant, a cationic surfactant, and a bridging surfactant was prepared by mixing at room temperature approximately equal molar quantities of Alpha Step® ML-40, QC_{10,} and Amphosol® CA. A 33.03% clear liquid phase free of precipitate was obtained. This surfactant blend displayed remarkable synergism, as shown in Table II, which shows that the ternary surfactant blend possesses surface tension, wetting, and foaming properties all dramatically better than the properties of any single surfactant or combination of two surfactants.

TABLE II

| Surfactant | Surface Tension | Wetting | Foaming |
|---|---|---|---|
| System | (mN/m) | (s) | (Initial/Final)(cm) |
| Alpha Step ® ML-40 | 26.92 | 81 | 12/11.8 |
| QC10 | 39.28 | 100 | 0/0 |
| Amphosol ® CA | 36.75 | 100 | 13/12.9 |
| QC10/ | 34.67 | 100 | 15/14.5 |
| Amphosol ® CA | | | |
| Alpha Step ® ML-40/ | 30.33 | 17 | 14.5/14.3 |
| Amphosol ® CA | | | |
| Alpha Step ® ML-40/ | ppt | ppt | ppt |
| QC10 | | | |
| Alpha Step ® ML-40/ | 26.11 | 4 | 15.7/15.7 |
| QC10/Amphosol ® CA | | | |

### EXAMPLE 3

Several ternary surfactant blends as shown in Table III were prepared by mixing at room temperature approximately equal moles of an anionic surfactant, a cationic surfactant, and a bridging surfactant. Appearance, surface tension, wetting, and foaming properties were evaluated for each blend over a range of pH. For the systems tested, when a betaine is used as the bridging surfactant, clarity can be maintained over a wide pH range. When an amine oxide is used as the bridging surfactant, clarity can be maintained when pH is above about 7.

TABLE III

| | | Appearance | Appearance | Surface | | Foaming |
|---|---|---|---|---|---|---|
| Surfactant | | (33-34% | (0.1% | Tension | Wetting | Initial/Final |
| System | pH | active) | active) | (mN/m) | (s) | (cm) |
| Alpha Step ®MC-48/ | 10.7 | clear | clear | 29.42 | 13 | 15.5/15.4 |
| QC8/Amphosol ® CA | 7.9 | clear | clear | 29.21 | 12 | 15.3/15.2 |
| | 5.5 | clear | clear | 29.62 | 15 | 15.1/15 |
| | 3.7 | clear | clear | 27.95 | 10 | 15.4/15.3 |
| | 2 | clear | clear | 26.94 | 14 | 15.3/15.2 |
| Alpha Step ®MC-48/ | 11.2 | clear | clear | 27.31 | 6 | 14.8/14.7 |
| QC8/Ammonyx ® LO | 7.6 | clear | clear | 26.65 | 6 | 14.4/14.2 |
| | 6 | clear | hazy | 25.69 | 19 | 10.7/10.5 |
| | 4 | clear | very hazy | 25.6 | 44 | 2.2/2 |
| | 2 | clear | precipitate | — | — | — |
| Polystep ®B-25/ | 10.8 | clear | clear | 27.26 | 5 | 16.9/16.8 |
| QC8/Amphosol ® CA | 7.7 | clear | clear | 27.73 | 5 | 17/16.9 |
| | 5.8 | clear | clear | 26.92 | 5 | 17.9/17.8 |
| | 4 | clear | clear | 26.87 | 6 | 17/16.9 |
| | 2 | clear | sl. hazy | 25.74 | 12 | 4/3.8 |
| Polystep ®B-25/ | 10.4 | clear | clear | 24.59 | 8 | 16/16 |
| QC8/Ammonyx ® LO | 8.8 | clear | clear | 24.35 | 3 | 15.5/15.5 |
| | 8 | clear | clear | 24.76 | 5 | 15.8/15.7 |
| | 6 | fl-white | sl. hazy | 24.98 | 33 | 9/8.8 |
| | 4 | 2 layers | — | — | — | — |
| | 2 | fl-white | precipitate | — | — | — |

### EXAMPLE 4

Several ternary surfactant blends as shown in Table IV were prepared by mixing at room temperature approximately equal moles of an anionic surfactant, a cationic surfactant, and a bridging surfactant. Appearance, surface tension, wetting, and foaming properties were evaluated for each blend. The results indicate that when magnesium ions are added to the ternary surfactant blends, the clarity and surfactant properties of the mixture are maintained.

TABLE IV

| | Appearance | | Surface | Wetting | Foaming |
|---|---|---|---|---|---|
| Surfactant | (34-35% | Appearance | Tension | (seconds | Initial/Final |
| System | active) | (0.1% active) | (mN/m) | (s)) | (cm) |
| Alpha ®Step MC-48/ | clear | clear | 29.62 | 15 | 15.1/15 |
| QC8/Amphosol ® CA | | | | | |
| Alpha ®Step MC-48/ | clear | clear | 28.73 | 15 | 15.1/15 |
| QC8/Amphosol ® CA + | | | | | |
| 1:1 mole ratio of | | | | | |
| Alpha ®Step MC-48: | | | | | |
| MgCl₂ | | | | | |
| Alpha ®Step MC-48/ | clear | clear | 28.51 | 12 | 15.1/15 |
| QC8/Amphosol ® CA + | | | | | |
| 1:0.5 mole ratio of | | | | | |
| Alpha ®Step MC-48: | | | | | |
| MgCl₂ | | | | | |
| Alpha ®Step MC-48/ | clear | clear | 26.65 | 6 | 14.4/14.2 |
| QC8/Ammonyx ® LO | | | | | |
| Alpha ®Step MC-48/ | clear | clear | 26.65 | 6 | 15.5/15.4 |
| QC8/Ammonyx ® LO + | | | | | |
| 1:1 mole ratio of | | | | | |
| Alpha ®Step MC-48: | | | | | |
| MgCl₂ | | | | | |
| Alpha ®Step MC-48/ | clear | clear | 26.94 | 6 | 15.5/15.4 |
| QC8/Ammonyx ® LO + | | | | | |
| 1:0.5 mole ratio of | | | | | |
| Alpha ®Step MC-48: | | | | | |
| MgCl₂ | | | | | |

### EXAMPLE 5

Several ternary surfactant blends as shown in Table V were prepared by mixing at room temperature approximately equal molar quantities of an anionic surfactant, a cationic surfactant, and a bridging surfactant. Surface tension, wetting, and foaming properties were evaluated for each blend. The results indicate that surfactant properties of the ternary blends can be tailored by altering the identity of the bridging surfactant, or the chain length of the anionic and/or cationic surfactant. For example, increasing the chain length of the cationic surfactant decreases wetting time (12 seconds, compared to 5 seconds). Furthermore, increasing the chain length of the anionic surfactant also decreases wetting time (7 seconds, as compared to 3 seconds). Finally, changing the bridging surfactant from an amine oxide to a betaine increases foaming (14.7 cm., compared to 17.3 cm.).

TABLE V

| | | | Foaming |
|---|---|---|---|
| Surfactant | Surface Tension | Wetting | (Initial/Final) |
| System | (mN/m) | (s) | (cm) |
| Bioterge ® PAS-8S/ | 25.72 | 12 | 14.7/14.6 |
| QC8/Ammonyx ® LO | | | |
| Bioterge ® PAS-8S/ | 26.58 | 5 | 15.7/15.5 |
| DTMAB/Ammonyx ® LO | | | |
| Polystep ® B-29/ | 24.88 | 7 | 14.7/14.6 |
| QC8/Ammonyx ® LO | | | |
| Polystep ® B-25/ | 24.35 | 3 | 15.5/15.5 |
| QC8/Ammonyx ® LO | | | |
| Polystep ® B-25/ | 27.73 | 5 | 17.0/16.9 |
| QC8/Amphosol ® CA | | | |

### EXAMPLE 6

Several ternary surfactant blends as shown in Table VI were prepared by mixing at room temperature approximately equal molar quantities of an anionic surfactant, a cationic surfactant, and a bridging surfactant Appearance, surface tension, wetting, and foaming properties were evaluated for each blend. The results indicate that clarity and surfactant properties of the ternary blends of the invention can be maintained for a range of anionic, cationic, and bridging surfactants.

TABLE VI

| | | | Surface | | Foaming |
|---|---|---|---|---|---|
| Surfactant | Appearance | Appearance | Tension | Wetting | Initial/ |
| System | (% active) | (0.1% active) | (mN/m) | (s) | Final (cm) |
| Bioterge ®PAS-8S/ | clear (34.42) | clear | 24.37 | 14 | 13.0/12.6 |
| DTMAB/Ammonyx ® C8 | | | | | |
| Maprosyl ®30/ | clear (31.79) | clear | 27.56 | 4 | 13.7/13.5 |
| DTMAB/Ammonyx ® C8 | | | | | |
| Stepan-Mild ®SL3/ | clear (31.93) | clear | 29.10 | 11 | 13.2/13.1 |
| DTMAB/Ammonyx ® C8 | | | | | |
| Bioterge ®PAS-8S/ | clear (32.28) | clear | 27.41 | — | — |
| Cetac 30/Ammonyx ® C8 | | | | | |
| Stepan-Mild ®SL3/ | clear (30.62) | clear | 26.97 | — | — |
| Cetac 30/Ammonyx ® C8 | | | | | |
| Bioterge ®PAS-8S/ | clear (33.77) | clear | 25.72 | 12 | 14.7/14.6 |
| QC8/Ammonyx ® LO | | | | | |
| Alpha Step ® MC48/ | clear (34.44) | clear | 26.65 | 6 | 14.4/14.2 |
| QC8/Ammonyx ® LO | | | | | |
| Maprosyl ®30/ | clear (31.21) | clear | 28.66 | 5 | 15.5/15.4 |
| QC8/Ammonyx ® LO | | | | | |
| Bioterge ®AS-40/ | clear (34.52) | clear | 27.88 | 9 | 14.2/14.0 |
| QC8/Ammonyx ® LO | | | | | |
| Stepan-Mild ®SL3/ | clear (31.48) | clear | 27.88 | 12 | 14.5/14.3 |
| QC8/Ammonyx ® LO | | | | | |
| Alpha Step ®ML-40/ | clear (33.25) | clear | 25.69 | 4 | 15.1/15.0 |
| QCB/Ammonyx ® LO | | | | | |
| Polystep ®B-29/ | clear (32.16) | clear | 24.88 | 7 | 14.7/14.6 |
| QC8/Ammonyx ® LO | | | | | |
| Polystep ®B-25/ | clear (34.05) | clear | 24.35 | 3 | 15.5/15.5 |
| QC8/Ammonyx ® LO | | | | | |
| Bioterge ®PAS-8S/ | clear (33.39) | clear | 30.99 | 46 | 14.5/14.5 |
| QC8/Amphosol ® CA | | | | | |
| Alpha Step ®MC-48/ | clear (34) | clear | 29.62 | 15 | 15.1/15.0 |
| QC8/Amphosol ® CA | | | | | |
| Maprosyl ®S30/ | clear (31.2) | clear | 30.89 | 15 | 14.8/14.7 |
| QC8/Amphosol CA | | | | | |
| Bioterge ®AS-40/ | clear (34.06) | clear | 29.96 | 15 | 14.1/14.0 |
| QC8/Amphosol CA | | | | | |
| Stepan-Mild ®SL3/ | clear (31.45) | clear | 30.84 | 29 | 13.8/13.8 |
| QC8/Amphosol CA | | | | | |
| Alpha Step ML-40/ | clear (32.97) | clear | 28.73 | 8 | 15.9/15.8 |
| QC8/Amphosol ® CA | | | | | |
| Stepanol ®WA-Extra/ | clear (30.76) | clear | 27.21 | 15 | 3.0/2.9 |
| QC8/Amphosol ® CA | | | | | |
| Polystep ®B-29/ | clear (32.02) | clear | 30.67 | 29 | 15.6/15.5 |
| QC8/Amphosol ® CA | | | | | |
| Polystep ®B-25/ | clear (33.64) | clear | 27.73 | 5 | 17.0/6.9 |
| QC8/Amphosol ® CA | | | | | |
| Steol ®CS-370/ | clear (42.3) | clear | 26.55 | 19 | 10.3/10.0 |
| DTMAB/Ammonyx ® LO | | | | | |
| Steol ®CS-460/ | clear (45.01) | clear | 26.28 | 22 | 7.4/7.3 |
| DTMAB/Ammonyx ® LO | | | | | |
| Bioterge ®PAS-8S/ | clear (31.89) | clear | 28.12 | 10 | 14.5/14.4 |
| Cetac30/Ammonyx ® LO | | | | | |
| Polystep ®B-20/ | clear (29.95) | clear | 37.44 | — | — |
| Cetac30/Ammonyx ® LO | | | | | |
| Polystep ®B-22/ | clear (29.84) | clear | 30.16 | — | — |
| Cetac30/Ammonyx ® LO | | | | | |

### EXAMPLE 7

Various surfactant blends were prepared as shown in Table VII at approximately 1:1:1 molar ratios of the various components. The appearance of each blend as a concentrated (33-38%) and dilute (0.1-1.0%) composition was noted. These blends comprised multiple anionic surfactants and were clear upon the addition of an amine oxide, demonstrating that the bridging surfactant promotes solubility of anionic-cationic complexes even in mixed anionic systems.

TABLE VII

| | Appearance | Appearance |
|---|---|---|
| Surfactant System | (concentrated) | (diluted) |
| Steol ® CS-460/ | clear | clear |
| Alpha Step ® MC-48/ | | |
| DTMAB/Ammonyx ® LO | | |
| Maprosyl ® 30/ | clear | clear |
| Alpha Step ® MC-48/ | | |
| DTMAB/Ammonyx ® LO | | |
| Polystep ® B-20/ | clear | clear |
| Alpha Step ® MC-48/ | | |
| DTMAB/Ammonyx ® LO | | |
| Polystep ® B-22/ | clear | clear |
| Alpha Step ® MC-48/ | | |
| DTMAB/Ammonyx ® LO | | |

### EXAMPLE 8

Various concentrated (30-40%) surfactant blends were prepared as shown in Table VIII at approximately 1:1:1 molar ratios of the components. The appearance of each blend was noted. None of the concentrated blends in Table VIII that comprised only anionic and cationic surfactants were flowable. Surprisingly, however, the addition of an amine oxide, ethoxamide, and/or betaine bridging surfactant to the anionic-cationic blends rendered a final composition that was flowable. This observation demonstrates that the present invention allows for production of flowable concentrated surfactant blends comprising anionic, cationic, and bridging surfactants

TABLE VIII

| | Appearance |
|---|---|
| Surfactant System | (concentrated) |
| Stepanol ® WA-Extra/QC10 | paste |
| Stepanol ® WA-Extra/QC10/Ammonyx ® C8 | flowable |
| Stepanol ® WA-Extra/QC10/Ammonyx ® C8/ | flowable |
| Amphosol ® CA | |
| Stepanol ® WA-Extra/QC10/Ammonyx ® MCO | flowable |
| Stepanol ® WA-Extra/QC10/Ammonyx ® LO | flowable |
| Stepanol ® WA-Extra/QC10/Ammonyx ® C10 | flowable |
| Stepan-Mild SL3 ® /Cetac 30 | paste |
| Stepan-Mild SL3 ® /Cetac 30/Amidox ® C-5 | flowable |
| Stepan-Mild SL3 ® /Cetac 30/Ammonyx ® C8 | flowable |

### EXAMPLE 9

Various concentrated (30-40%) surfactant blends were prepared as shown in Table IX and Table X. Each blend was prepared at an approximately 1:1:1 molar ratio of the components. The appearance of each blend was noted. The concentrated blends in Table IX, which comprised anionic, cationic, and bridging surfactants, were flowable. The concentrated blends in Table X, presented for comparison purposes, were not flowable even though these blends comprised anionic, cationic, and bridging surfactants and were prepared according to the same procedure used in preparing the blends in Table IX. This observation demonstrates that not every combination of anionic, cationic, and bridging surfactant is flowable. However, routine screening of combinations of anionic, cationic, and bridging surfactants allows determining which ternary surfactant blends are flowable.

TABLE IX

| | Appearance |
|---|---|
| Surfactant System | (concentrated) |
| Maprosyl ® 30/DTMAB/Ammonyx ® C8 | flowable |
| Stepan-Mild ® SL3/QC10/Amidox ® C-5 | flowable |
| Bioterge ® PAS-8S/DTMAB/Ammonyx ® C8 | flowable |
| Alpha Step ® MC-48/DTMAB/Ammonyx ® C8 | flowable |
| Steol ® CS-370/Cetac 30/Ammonyx ® C8 | flowable |

TABLE X

| | Appearance |
|---|---|
| Surfactant System | (concentrated) |
| Maprosyl ® 30/Cetac 30/Ammonyx ® LO | paste |
| Maprosyl ® 30/Cetac 30/Ammonyx ® MCO | paste |
| Stepan-Mild ® SL3/Cetac 30/Ammonyx ® LO | paste |
| Stepan-Mild ® SL3/Cetac 30/Ammonyx ® MCO | paste |
| Bioterge ® PAS-8S/Cetac 30/Ammonyx ® MCO | paste |
| Bioterge ® PAS-8S/DTMAB/Ammonyx ® MCO | paste |
| Bioterge ® PAS-8S/Cetac 30/Amphosol ® CA | paste |
| Alpha Step ® MC-48/Cetac 30/Ammonyx ® MCO | paste |
| Alpha Step ® MC-48/DTMAB/Ammonyx ® MCO | paste |
| Steol ® CS-370/QC10/Ammonyx LO | paste |

### EXAMPLE 10

### Antimicrobial Compositions, Preparation and Evaluation

Antimicrobial compositions of the invention are prepared as follows. The compositions were prepared by mixing at room temperature (25° C.) approximately equal molar amounts of anionic, cationic, and bridging surfactants. Each concentrated composition, i.e., having a concentration of about at least 25% by weight, was clear and flowable. Compositions according to the invention are shown in Table XI.

TABLE XI

| | FORMULATION | | | | | | | |
|---|---|---|---|---|---|---|---|---|
| | (weight percent active components) | | | | | | | |
| component | A1 | A2 | A3 | A4 | A5 | A7 | A8 | A9 |
| Ammonyx-LO | 11.8 | 10.38 | 11.04 | 10.81 | | | 11.63 | 11.29 |
| Amphosol CA | | | | | 13.8 | 13.44 | | |
| BIO-TERGE PAS- | 9.59 | | | | | | 11.19 | 10.86 |
| 8S | | | | | | | | |
| Bioterge AS-40 | | | | | | 11.69 | | |
| BTC 2125 M | | | | 16.53 | | | | 17.27 |
| BTC 65NF | 16.49 | 14.49 | 15.41 | | 13.36 | 13.01 | 16.25 | |
| Alpha-step MC48 | | 13.98 | | | | | | |
| Steol CS 460 | | | 19.38 | 18.99 | 16.81 | | | |
| Total weight percent | 37.89 | 38.85 | 45.83 | 46.33 | 43.97 | 38.14 | 39.07 | 39.43 |
| Actives | | | | | | | | |
| interfacial tension | NT | NT | NT | NT | 0.01 | 0.08 | NT | NT |
| against isopropyl | | | | | | | | |
| palmitate (dynes/ | | | | | | | | |
| cm) | | | | | | | | |
| appearance upon | hazy | NT | hazy | NT | NT | NT | hazy | hazy |
| dilution | | | | | | | | |

| | |
|---|---|
| Rheology | all flowable |

NT: not tested

Each of these formulation was prepared essentially according to the above procedure.

It is noted that formulations of approximately 1:1 molar quantities of (i) PAS-8S and BTC 65NF and (ii) CS 460 and BTC 65NF separated into two layers upon standing. On the other hand, all of Formulations A1-A9 were stable, clear and flowable one phase systems.

Formulation A1 was tested as described below and compared with the antimicrobial activity of the individual components of the mixture (Formulations C1-C3). The concentrations of the Formulation C1-C3 are given in the following table.

| | Formulation | | |
|---|---|---|---|
| component | C1 | C2 | C3 |
| AMMONYX-LO | 29.92 | | |
| (Lauramine oxide) | | | |
| BIO-TERGE PAS-8S | | 34.33 | |
| (Sodium 1-octane | | | |
| sulfonate) | | | |
| BTC 65NF (N-Alkyl | | | 50.58 |
| dimethyl benzyl ammonium | | | |
| chloride) | | | |
| Total weight percent | 29.92 | 34.33 | 50.58 |
| Actives | | | |

Formulations A1 and C1-C3 evaluated according to the following procedure.

| | | |
|---|---|---|
| Method Used: | | Disinfection and Sterilization, Second |
| | | Edition By G. Sykes, M.Sc. (Lond) |
| | | Hon. M.P.S., F.R.I.C. |
| | | Methods of Testing Antiseptics |
| | | “Bacteriostatic Tests” |
| Organisms: | Bacteria: | Staphylococcus aureus (ATCC 6538) |
| | | Escherichia coli (ATCC 11229) |
| | | Pseudomonas aeruginosa (ATCC 15442) |
| | Fungi: | Aspergillus niger (ATCC 16404) |
| | | Candida albicans (ATCC 10231) |

### Preparation of Test Inoculum

| | |
|---|---|
| Bacteria: | Dilute 24 ± 1 hour old culture of bacterium 1:10 in Nutrient |
| | Broth |
| Fungi: | Dilute 24 ± 1 hour old culture of Candida albicans or 7- |
| | 10 day old culture of Aspergillus fumigatus in 0.85% sterile |
| | saline to obtain a cell/spore suspension containing |
| | 10⁷ spores or cells/ml. |

### Test Procedure

Add 0.1 ml of inoculum to each 20×150 mm test tube containing 9.0 ml of Nutrient or Sabouraud Dextrose Broth and 1 ml of a aqueous dilution of the test sample.

Vortex tube to mix then incubate bacteria inoculated tubes at 36±1° C. for a minimum of 48±1 hour and fungi inoculated tubes at 28° C. for 7-10 days. Following incubation, observe tubes visually for the presence of growth (i.e. turbidity). Perform all testing in triplicate.

### Test Results

Bacteriostatic and fungistatic responses for each test sample are provided in Table 9-I. Minimum Inhibitory Concentration(MIC) values derived from these responses are shown in Table 10-II.

The MIC values listed in Table 10-II represent the lowest concentration of the test sample which completely inhibited growth of the test organism, based on visual observations, in each of the three replicate tests.

TABLE 10.1
Minimum Inhibitory Concentration² Test
Bacteriostatic/Fungistatic Responses

| Sample | Conc. | Test Organism vs. Observations | | | | |
|---|---|---|---|---|---|---|
| ID | (ppm, active) | S. aureus | E. coli | P. aeruginosa | A. niger | C. albicans |
| AMMONYX-LO | 1 | +++ | +++ | +++ | +++ | +++ |
| | 10 | +++ | +++ | +++ | +++ | +++ |
| | 100 | 000 | ±±± | +++ | 000 | 000 |
| | 1000 | 000 | 000 | ±±± | 000 | 000 |
| BIO-TERGE PAS- | 1 | +++ | +++ | +++ | +++ | +++ |
| 8S | 10 | +++ | +++ | +++ | +++ | +++ |
| | 100 | +++ | +++ | +++ | +++ | +++ |
| | 1000 | +++ | +++ | +++ | +++ | +++ |
| BTC 65NF | 1 | 000 | +++ | +++ | +++ | +++ |
| | 10 | 000 | 000 | +++ | 000 | 000 |
| | 100 | 000 | 000 | 000 | 000 | 000 |
| | 1000 | 000 | 000 | 000 | 000 | 000 |
| Formulation A1 | 1 | +++ | +++ | +++ | +++ | +++ |
| | 10 | 000 | 000 | +++ | +++ | ±±± |
| | 100 | 000 | 000 | 000 | 000 | 000 |
| | 1000 | 000 | 000 | 000 | 000 | 000 |

²Key:
+= Growth Observed
0= No Growth Observed
±= Slight Growth Obs rved
Testing performed in triplicate
Note:
The Minimum Inhibitory Concentration (MIC) value represents the lowest concentration of the test substance which completely inhibits growth in all replicates.

TABLE 10-II
Summary of Minimum Inhibitory
Concentration Test Results¹

| | Test Organism vs. Minimum Inhibitory Concentration | | | | |
|---|---|---|---|---|---|
| Sample | (ppm, active) | | | | |
| ID | S. aureus | E. coli | P. aeruginosa | A. niger | C. albicans |
| AMMONYX-LO | 100 | 1000 | >1000 | 100 | 100 |
| BIO-TERGE PAS-8S | >1000 | >1000 | >1000 | >1000 | >1000 |
| BTC65NF | <1 | 10 | 100 | 10 | 10 |
| A1* | 10 | 10 | 100 | 100 | 100 |

*

| | Concentration in PPM of Components Present | |
|---|---|---|
| | in A1 at MIC Concentrations of 10 and 100 ppm, | |
| | active | |
| Ingredient | 10 | 100 |
| AMMONYX-LO | 3.115 | 31.150 |
| BIO-TERGE PAS-8S | 2.532 | 25.320 |
| BTC 65NF | 4.353 | 43.530 |

¹MIC values shown are based upon the bacteriostatic/fungistatic responses shown in Table 9-I

### EXAMPLE 11

### Time-Kill

Formulation 11-1 was prepared as described above and evaluated at two different concentrations against various microorganisms to determine the time required to kill certain levels of the microorganisms. Control formulation 11-2 was also tested in this assay at the same concentrations.

The compositions of the various formulations are shown in the following tables.

| | |
|---|---|
| | Formulation 11-1 |
| Component | (active components, ppm) |

| | | |
|---|---|---|
| AMMONYX-LO | 14.888 | 29.776 |
| BIO-TERGE PAS-8S | 14.321 | 28.641 |
| BTC 65NF | 20.792 | 41.583 |
| total concentration, ppm | 50 | 100 |

| | |
|---|---|
| | Formulation 11-2 |
| Component | (active components, ppm) |

| | | |
|---|---|---|
| AMMONYX-LO | 29.997 | 59.994 |
| BIO-TERGE PAS-8S | 24.512 | 49.023 |
| BTC 65NF | 0 | 0 |
| total concentration, ppm | 50 | 100 |

### Test Organisms (Base Set):

Staphylococcus aureus (ATCC 6538) “Sa”

Escherichia coli (ATCC 11229) “Ec”

Psudomonas aeruginosa (ATCC 15442) “Pa”

Aspergillus niger (ATCC 6275) “An”

Candida albicans (ATCC 10231)“Ca”

Preparation: Transfer each organism from stock slant to sterile Nutrient Broth (NB) tube. Incubate NB tubes at 36±1° C. After 24 hours incubation, subculture each broth culture to fresh tube of sterile NB. Repeat subculturing procedure at 24 hour intervals. Use cultures for testing after at least three consecutive 24 hour subculturings.

### Procedure

1. Label sterile 25×150 mm tubes and 100×15 mm petri plates. Include: test substance name, date, contact period, dilution, and organism name.

2. Dispense 4.9 gram aliquots of each sample* into sterile medication tubes. Use one tube per organism. (*log₁₀ reductions can be calculated by subtracting the log₁₀ of the average inoculum count from the log₁₀ of the test substance count after 30 seconds, 1 minute or 5 minutes contact)

3. Centrifuge 18-24 hour broth culture of each test organism. Decant supernatant and re-suspend in equal volume of phosphate buffer. Re-centrifuge and re-suspend in phosphate buffer. Prepare 1:10 dilution of each culture suspension by adding 2 ml of culture to 18 ml of phosphate buffer. Mix by vortexing. Use this 1:10 dilution as the test inoculum.

4. Perform initial plate count of the test inoculum by adding 0.1 ml of the inoculum to 4.9 grams of sterile distilled water. Vortex to mix, then remove 1 ml of solution and dilute in D/E Broth to 1:100,000. Plate 1:1,000, 1:10,000, and 1:100,000 dilutions in Microbial Content Agar (Difco).

5. Begin testing of substances by adding 0.1 ml of test inoculum to sample tube. Vortex to mix. After 30 seconds, remove 1 ml of sample and add to 9 mls of DE broth, rinse pipette 5 times with sample and vortex. Remove additional 1 ml sample after 1 and 5 minutes contact and add to 9 mls of D/E Broth. Dilute each tube to 1:10,000 in D/E Broth and plate 1:1,000, 1:10,000, and 1:100,000 dilutions in Microbial Content Agar.

6. Repeat step 5 for all samples and test organisms.

7. After completion of step 6, perform final plate count of inoculum suspension by adding 0.1 ml of the suspension to 4.9 grams of sterile distilled water. Vortex to mix then remove 1 ml of solution and dilute in D/E Broth to 1:100,000. Plate 1:1,000, 1:10,000, and 1:100,000 dilutions in Microbial Content Agar (Difco).

8. Incubate plates at 36±1° C. for 48 hours.

9. Record all test results and calculate percent kills* after 30 seconds, 1 minute, and 5 minutes contact as follows: (*log₁₀ reductions can be calculated by subtracting the log₁₀ of the average inoculum count from the log₁₀ of the test substance count after 30 seconds, 1 minute or 5 minutes contact)

$$\frac{X - Y}{X} \times 100 \% = \% kill$$

where:

X=Average of initial and final inoculum control counts, org/ml (Note: The difference between initial and final inoculum counts must be≦1 log for a valid study)

Y=Test substance bacterial count as org/ml after 30 seconds, 1 minute, or 5 minutes contact

The results of this time-kill experiment are shown below in Tables 11-I, 11-II, and 11-III.

TABLE 11 - I

| Active Test | | Contact Time vs. Percent Kill | | |
|---|---|---|---|---|
| Concentration | | (% K) | | |
| BTC 65NF | | 30 Seconds | 60 Seconds | 5 Minutes |
| (ppm) | Test Organism | % K | % K | % K |
| 100 | Sa* | 65.5 | 83.6 | >99.8 |
| | Ec | >99.9 | >99.9 | >99.9 |
| | Pa | 99.9 | 99.9 | 99.9 |
| | An* | 0.0 | 22.5 | 92.3 |
| | Ca* | >99.7 | >99.7 | >99.7 |
| 50 | Pa* | 99.9 | >99.9 | >99.9 |
| | (ATCC 9027) | | | |

TABLE 11 - II

| Active Test | | | | |
|---|---|---|---|---|
| Concentration | | Contact Time vs. Percent Kill | | |
| Formu- | | (% K) | | |
| lation 11-2) | | 30 Seconds | 60 Seconds | 5 Minutes |
| (ppm) | Test Organism | % K | % K | % K |
| 100 | An* | 0.0 | 0.0 | 0.0 |
| | Ca* | 0.0 | 0.0 | 33.3 |
| 50 | Pa* | 95.9 | >99.9 | >99.9 |
| | (ATCC 9027) | | | |

TABLE 11 - III

| Active Test | | | | |
|---|---|---|---|---|
| Concentration | | Contact Time vs. Percent Kill | | |
| Formu- | | (% K) | | |
| lation 11-1 | | 30 Seconds | 60 Seconds | 5 Minutes |
| (ppm) | Test Organism | % K | % K | % K |
| 100 | Sa* | 91.5 | 97.8 | >99.8 |
| | An* | 0.0 | 0.0 | 84.7 |
| | Ca* | 94.9 | 97.2 | >99.7 |
| 50 | Pa* | 99.8 | >99.9 | >99.9 |
| | (ATCC 9027) | | | |

The invention and the manner and process of making and using it, are now described in such full, clear, concise and exact terms as to enable any person skilled in the art to which it pertains, to make and use the same. Although the foregoing describes preferred embodiments of the present invention, modifications may be made therein without departing from the spirit or scope of the present invention as set forth in the claims. To particularly point out and distinctly claim the subject matter regarded as invention, the following claims conclude this specification.

What is claimed is:

1. A surfactant blend comprising:
(a) an antimicrobial compound of the formula: 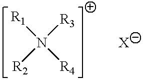
wherein
R₁ and R₂ are straight or branched chain lower alkyl groups having from one to seven carbon atoms;
R₃ is a straight or branched chain higher alkyl group having from about eight to twenty carbon atoms, or a benzyl group optionally substituted with C₁-C₆ alkyl;
R₄ is a straight or branched chain higher alkyl group having from about eight to twenty carbon atoms; and
X is an anion forming a water soluble salt, such as, halogen, methosulfate, saccharinate, sulfate, ethosulfate, tosylate, acetate, phosphate, nitrate, sulfonate, or carboxylate;
(b) an anionic surfactant;
(c) a bridging surfactant.

2. An antimicrobial composition comprising water and an amount of a blend according to claim 1 effective to control the growth of microorganisms in contact with the composition.

3. An antimicrobial composition comprising water and an amount of a blend according to claim 1 effective to produce a concentration of the anti-microbial compound of from about 1 to about 3000 ppm.

4. A blend according to claim 1, wherein the anionic surfactant is selected from the group consisting of alkyl sulfates, alkyl ether sulfates, and alkyl sulfonates.

5. A blend according to claim 4, wherein the anionic surfactant is an alkyl sulfonate or alkyl sulfate having from about 8 to about 10 carbon atoms.

6. A blend accord to claim 5, wherein the bridging surfactant is an amine oxide or an amphoteric surfactant.

7. A method for controlling the growth of microorganisms, comprising contacting a surface suspected of containing microorganisms with a blend according to claim 1.

8. A surfactant blend comprising:
(a) a quaternary ammonium compound of the formula: 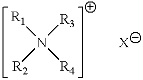
wherein
R₁ and R₂ are straight or branched chain lower alkyl groups having from one to seven carbon atoms;
R₃ is a straight or branched chain higher alkyl group having from about eight to twenty carbon atoms, or a benzyl group optionally substituted with C₁-C₆ alkyl;
R₄ is a straight or branched chain higher alkyl group having from about eight to twenty carbon atoms; and
X is an anion forming a water soluble salt;
(b) an anionic surfactant which is
(i) an alkyl sulfate having an average of from about 8 to about 16 carbon atoms;
(ii) an alkyl sulfonate having an average of from about 8 to about 18 carbon atoms;
(iii) an alkyl ether sulfate having an average of from about 8 to about 16 carbon atoms in the alkyl portion and from about 1 to about 30 moles of ethylene oxide;
(iv) an α-olefin sulfonate having an average of from about 12 to about 18 carbon atoms;
(v) an α-sulfonated C₁-C₆ alkyl ester of a fatty acid having an average of from about 11 to about 16 carbon atoms;
(vi) a sulfosuccinate having an average of from about 10 to about 16 carbon atoms;
(vii) a sarcosinate having an average of from about 10 to about 16 carbon atoms; or
(viii) a sulfoacetate having an average of from about 12 to about 20 carbon atoms;
or mixtures thereof;
(c) a bridging surfactant selected from the group consisting of amine oxides, ethoxamides, and betaines;
optionally (d) a cationic surfactant which is a quaternary ammonium compound of the formula: 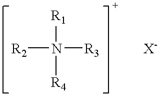
where
R₁, R₂, and R₃ are independently ethyl or methyl;
R₄ is an alkyl group having an average of from about 8 to about 16 carbon atoms; and
X is halogen, sulfate, methosulfate, ethosulfate, tosylate, acetate, phosphate, nitrate, sulfonate, or carboxylate;
wherein the total concentration of combined quaternary ammonium compound, anionic, and bridging surfactants is from about 30 to about 80 percent by weight, and wherein the surfactant blend is flowable.

9. An aqueous liquid phase comprising the blend of claim 8, wherein the cationic surfactant, anionic surfactant, and bridging surfactant are each present in an amount of from about 5 to about 35 percent by weight.

10. A blend according to claim 8, wherein the anionic surfactant is an alkyl sulfate having an average of from about 10 to about 12 carbon atoms.

11. A blend according to claim 8, wherein the anionic surfactant is an α-sulfonated C₁-C₆ alkyl ester of a fatty acid having an average of from about 11 to about 16 carbon atoms.

12. A blend according to claim 8, wherein the anionic surfactant is an alkyl sulfonate having an average of about 8 carbon atoms.

13. A blend according to claim 8, wherein the anionic surfactant is an alkyl ether sulfate having an average of from about 8 to about 16 carbon atoms in the alkyl portion and from about 1 to about 30 moles of ethylene oxide.

14. A method for preparing an antimicrobial composition comprising combining:
(a) a quaternary ammonium compound of the formula: 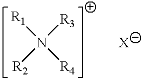
wherein
R₁ and R₂ are straight or branched chain lower alkyl groups having from one to seven carbon atoms;
R₃ is a straight or branched chain higher alkyl group having from about eight to twenty carbon atoms, or a benzyl group optionally substituted with C₁-C₆ alkyl;
R₄ is a straight or branched chain higher alkyl group having from about eight to twenty carbon atoms; and
X is an anion forming a water soluble salt;
optionally (d) a cationic surfactant which is a quaternary ammonium compound of the formula: 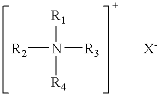
where
R₁, R₂, and R₃ are independently ethyl or methyl;
R₄ is an alkyl group having an average of from about 8 to about 16 carbon atoms; and
X is halogen, sulfate, methosulfate, ethosulfate, tosylate, acetate, phosphate, nitrate, sulfonate, or carboxylate; and
(b) an anionic surfactant which is
(i) an alkyl sulfate having an average of from about 8 to about 16 carbon atoms;
(ii) an alkyl sulfonate having an average of from about 8 to about 18 carbon atoms;
(iii) an alkyl ether sulfate having an average of from about 8 to about 16 carbon atoms in the alkyl portion and from about 1 to about 30 moles of ethylene oxide;
(iv) an α-olefin sulfonate having an average of from about 12 to about 18 carbon atoms;
(v) an α-sulfonated C₁-C₆ alkyl ester of a fatty acid having an average of from about 11 to about 16 carbon atoms;
(vi) a sulfosuccinate having an average of from about 10 to about 16 carbon atoms;
(vii) a sarcosinate having an average of from about 10 to about 16 carbon atoms; or
(viii) a sulfoacetate having an average of from about 12 to 20 carbon atoms;
or mixtures thereof; and
(c) a bridging surfactant selected from the group consisting of amine oxides, ethoxamides, and betaines;
wherein the total concentration of combined quaternary ammonium compound, anionic, and bridging surfactants is from about 30 to about 80 percent by weight, and wherein the surfactant blend is flowable.

15. An antimicrobial composition comprising:
(a) a quaternary ammonium compound of the formula: 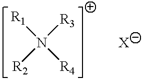
wherein
R₁ and R₂ are straight or branched chain lower alkyl groups having from one to seven carbon atoms;
R₃ is a straight or branched chain higher alkyl group having from about eight to twenty carbon atoms, or a benzyl group optionally substituted with C₁-C₆ alkyl;
R₄ is a straight or branched chain higher alkyl group having from about eight to twenty carbon atoms; and
X is an anion forming a water soluble salt;
(b) an anionic surfactant which is
(i) an alkyl sulfate having an average of from about 8 to about 16 carbon atoms;
(ii) an alkyl sulfonate having an average of from about 8 to about 18 carbon atoms;
(iii) an alkyl ether sulfate having an average of from about 8 to about 16 carbon atoms in the alkyl portion and from about 1 to about 30 moles of ethylene oxide;
(iv) an α-olefin sulfonate having an average of from about 12 to about 18 carbon atoms;
(v) an α-sulfonated C₁-C₆ alkyl ester of a fatty acid having an average of from about 11 to about 16 carbon atoms;
(vi) a sulfosuccinate having an average of from about 10 to about 16 carbon atoms;
(vii) a sarcosinate having an average of from about 10 to about 16 carbon atoms; or
(viii) a sulfoacetate having an average of from about 12 to about 20 carbon atoms;
or mixtures thereof;
(c) a bridging surfactant selected from the group consisting of amine oxides, ethoxamides, and betaines; and
optionally (d) a cationic surfactant which is a quaternary ammonium compound of the formula: 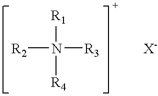
where
R₁, R₂, and R₃ are independently ethyl or methyl;
R₄ is an alkyl group having an average of from about 8 to about 16 carbon atoms; and
X is halogen, sulfate, methosulfate, ethosulfate, tosylate, acetate, phosphate, nitrate, sulfonate, or carboxylate;
wherein the total concentration of combined quaternary ammonium compound, anionic, and bridging surfactants is from about 0.1 to about 30 percent by weight, and wherein the surfactant blend is flowable.

16. A composition according to claim 15, wherein the anionic surfactant is an alkyl sulfate having an average of from about 10 to about 12 carbon atoms.

17. A composition according to claim 15, wherein the anionic surfactant is an α-sulfonated C₁-C₆ alkyl ester of a fatty acid having an average of from about 11 to about 16 carbon atoms.

18. A composition according to claim 15, wherein the anionic surfactant is an alkyl sulfonate having an average of about 8 carbon atoms.

19. A composition according to claim 18, wherein the anionic surfactant is an alkyl ether sulfate having an average of from about 8 to about 16 carbon atoms in the alkyl portion and from about 1 to about 30 moles of ethylene oxide.

20. An aqueous composition comprising water and the composition of claim 15, where the concentration of the quaternary ammonium compound in the composition is from about 1-3000 ppm.

21. An antimicrobial composition according to claim 15, wherein the amount of the blend of claim 1 is effective to produce a concentration of the anti-microbial compound of from about 1 to about 10 ppm.

22. A composition according to claim 1, further comprising a cationic surfactant of the formula: 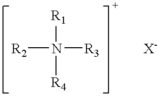
where
R₁, R₂, and R₃ are independently ethyl or methyl;
R₄ is an alkyl group having an average of from about 8 to about 16 carbon atoms; and
X is halogen, sulfate, methosulfate, ethosulfate, tosylate, acetate, phosphate, nitrate, sulfonate, or carboxylate.
